SEQUENCE LISTING (8 sequences)

SEQ ID NO: 1 - DNA, 122 bp, Artificial Sequence
  Description of Artificial Sequence Synthetic polynucleotide
ctcccgcagaacaccatgcgctccacggaagcaggtcctcactttaatgctgtggccactgagaagtattaaagtgaggacctgcttctgaggagcgccttgacagcagccatgggagggcc

SEQ ID NO: 2 - DNA, 1406 bp, Artificial Sequence
  Description of Artificial Sequence Synthetic polynucleotide
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcaattcacgcgtccatggcttagaaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatcacgagactagcctcgagcggccgcaattcgaacgctgacgtcatcaacccgctccaaggaatcgcgggcccagtgtcactaggcgggaacacccagcgcgcgtgcgccctggcaggaagatggctgtgagggacagggagtggcgccctgcaatatttgcatgtcgctatgtgttctgggaaatcaccataaacgtgaaatgtctttggatttgggaatcttataagttctgtatgagaccacaccggtaccgagctctcccgcagaacaccatgcgctccacggaagcaggtcctcactttaatgctgtggccactgagaagtattaaagtgaggacctgcttctgaggagcgccttgacagcagccatgggagggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 3 - DNA, 1412 bp, Artificial Sequence
  Description of Artificial Sequence Synthetic polynucleotide
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcaattcacgcgtatagtcttctgcacagggcattctttttgcttcaggatgtttacaacatttgctgcccacttttcctaggtttcttgagacctctacaagagttggagttgacacttggggtactttcttggtgtaacgaactaatagcctgaaaaaaagaagtcatgtgttttcagcaaggcaagaaactgtctaacatagtagataaaacagagaacacttggccggaatcaactaagatgttgctatgttccattcatcatattatctccatctgcagagtagtgggttagtggagggtagaaaacattctcctgaacaactagttaaacttggctttgagttccacctgtaccacttgcataatcttgggaaagtgagttgcctaattcagtgacattaataaatttattaatttcttctttcaataaaacctggagagagcttcatatgtatcagcatatgctaaacttgaaagatacaagtagaaaatggaaggaaatatatctgactcaatagggatagttcaagggttaaattaaaagtagtaaagtattataattaatctgacatggtaccctctagcggccgcaattcgaacgctgacgtcatcaacccgctccaaggaatcgcgggcccagtgtcactaggcgggaacacccagcgcgcgtgcgccctggcaggaagatggctgtgagggacagggagtggcgccctgcaatatttgcatgtcgctatgtgttctgggaaatcaccataaacgtgaaatgtctttggatttgggaatcttataagttctgtatgagaccacaccggtaccgagctctcccgcagaacaccatgcgctccacggaagcaggtcctcactttaatgctgtggccactgagaagtattaaagtgaggacctgcttctgaggagcgccttgacagcagccatgggagggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 4 - DNA, 105 bp, Artificial Sequence
  Description of Artificial Sequence Synthetic polynucleotide
ctgcgcgctcgctcgctcactgaggccgcccgggcaaagcccgggcgtcgggcgacctttggtcgcccggcctcagtgagcgagcgagcgcgcagagagggagtg

SEQ ID NO: 5 - DNA, 570 bp, Homo sapiens
atagtcttctgcacagggcattctttttgcttcaggatgtttacaacatttgctgcccacttttcctaggtttcttgagacctctacaagagttggagttgacacttggggtactttcttggtgtaacgaactaatagcctgaaaaaaagaagtcatgtgttttcagcaaggcaagaaactgtctaacatagtagataaaacagagaacacttggccggaatcaactaagatgttgctatgttccattcatcatattatctccatctgcagagtagtgggttagtggagggtagaaaacattctcctgaacaactagttaaacttggctttgagttccacctgtaccacttgcataatcttgggaaagtgagttgcctaattcagtgacattaataaatttattaatttcttctttcaataaaacctggagagagcttcatatgtatcagcatatgctaaacttgaaagatacaagtagaaaatggaaggaaatatatctgactcaatagggatagttcaagggttaaattaaaagtagtaaagtattataattaatctgacatggtacc

SEQ ID NO: 6 - DNA, 219 bp, Artificial Sequence
  Description of Artificial Sequence Synthetic polynucleotide
aattcgaacgctgacgtcatcaacccgctccaaggaatcgcgggcccagtgtcactaggcgggaacacccagcgcgcgtgcgccctggcaggaagatggctgtgagggacagggagtggcgccctgcaatatttgcatgtcgctatgtgttctgggaaatcaccataaacgtgaaatgtctttggatttgggaatcttataagttctgtatgagaccac

SEQ ID NO: 7 - DNA, 127 bp, Artificial Sequence
  Description of Artificial Sequence Synthetic polynucleotide
gatctttttccctctgccaaaaattatggggacatcatgaagccccttgagcatctgacttctggctaataaaggaaatttattttcattgcaatagtgtgttggaattttttgtgtctctcactcg

SEQ ID NO: 8 - DNA, 130 bp, Artificial Sequence
  Description of Artificial Sequence Synthetic polynucleotide
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

(19) United States
(12) Patent Application Publication
(10) Pub. No.: US 2021/0361318 A1
(43) Pub. Date: Nov. 25, 2021

(54) CANNULA SYSTEM AND USE THEREOF

(71) Applicant: VOYAGER THERAPEUTICS, INC., Cambridge, MA (US)

(72) Inventors: Holger Patzke, Cambridge, MA (US); Steven M. Hersch, Cambridge, MA (US); Jenna Carroll Soper, Winchester, MA (US); Adrian Philip Kells, Upper Arlington, OH (US); Nicholas Boulis, Atlanta, GA (US); Donna T. Ward, Groton, MA (US)

(21) Appl. No.: 17/257,418

(22) PCT Filed: Jul. 2, 2019
(86) PCT No.: PCT/US2019/040222

§ 371 Date: Dec. 31, 2020

Related U.S. Application Data
(60) Provisional application No. 62/693,040, filed on Jul. 2, 2018.
(60) Provisional application No. 62/731,879, filed on Sep. 15, 2018.
(60) Provisional application No. 62/746,108, filed on Oct. 16, 2018.
(60) Provisional application No. 62/823,198, filed on Mar. 25, 2019.

(51) Int. Cl. A61B 17/34; A61B 90/30; C12N 15/113

(52) U.S. Cl. CPC A61B 17/3421; A61B 17/3401; A61B 17/3478; A61B 2017/00911; C12N 15/113; A61B 2017/3407; A61B 90/30

(57) ABSTRACT

The present disclosure relates to cannula systems for delivery of a therapeutic agent to parenchymal tissue of a target subject. In certain embodiments, the cannula system is for delivery of a therapeutic agent to parenchymal tissue in the spine of a target subject. In certain embodiments, the cannula system includes a cannula positioning guide (CPG) useful in the delivery of a therapeutic agent to a localized target tissue of a subject. The guide may be used in a surgical suite or during a surgical procedure.

### CROSS REFERENCE TO RELATED APPLICATIONS

This application claims the benefit of U.S. Provisional Patent Application No. 62/693,040, filed Jul. 2, 2018, entitled TREATMENT OF ALS AND DISORDERS ASSOCIATED WITH THE SPINAL CORD, and U.S. Provisional Patent Application No. 62/731,879, filed Sep. 15, 2018, entitled CANNULA SYSTEM AND USE THEREOF, and U.S. Provisional Patent Application No. 62/746,108, filed Oct. 16, 2018, entitled CANNULA SYSTEM AND USE THEREOF, and U.S. Provisional Patent Application No. 62/823,198, filed Mar. 25, 2019, entitled CANNULA POSITIONING GUIDE AND USES THEREOF, the contents of which are each incorporated herein by reference in their entirety.

### REFERENCE TO THE SEQUENCE LISTING

The present application is being filed along with a Sequence Listing in electronic format. The Sequence Listing is provided as a file entitled 20571062PCTSL.txt, created on Jul. 2, 2019, which is 7,392 bytes in size. The information in the electronic format of the sequence listing is incorporated herein by reference in its entirety.

### FIELD OF THE DISCLOSURE

The present disclosure relates to cannula systems for delivery of a therapeutic agent to parenchymal tissue of a target subject. In certain embodiments, the cannula system is for delivery of a therapeutic agent to parenchymal tissue in the spine of a target subject. In certain embodiments, the cannula system includes a cannula positioning guide (CPG) useful in the delivery of a therapeutic agent to a localized target tissue of a subject. The guide may be used in a surgical suite or during a surgical procedure.

### BACKGROUND

Cannulas are a surgical tool used to accurately deliver a therapeutic agent to a target area or tissue of a subject. For example, cannulas can be used to deliver therapeutic agents to regions of the central nervous system (CNS), such as the brain or the spine.

Optimizing cannula performance can be complicated by several factors, including, but not limited to, the depth and volume of required injection, fluidity and diffusion properties of the therapeutic agents, dimensions and composition of individual cannula components, anatomical sensitivity and accessibility of target areas for injection, complexity of surgical procedures and techniques (free-hand or using guide equipment), reflux and backflow mitigation, and reliability. Consequently, there is a need for improvements to cannula systems that address these factors. The present disclosure describes solutions which address the shortcomings of current cannula systems.

### SUMMARY

The present disclosure presents improved cannula designs which provide therapeutic advantages such as reduced backflow, improved cannula placement accuracy and flexibility, improved ease-of-use, and improved delivery control.

The present disclosure presents a cannula system which includes a proximal region and a distal region, and which also includes a delivery tube (i.e. cannula tube) which extends from the proximal region of the cannula system to the distal region of the cannula system. The delivery tube can be made from a medical-grade material. The delivery tube can be made from stainless steel, titanium, polyvinylchloride (PVC), polymethylmethacrylate (PMMA), polytetrafluoroethylene (PTFE) or a fused silica such as quartz silica. In certain embodiments, the delivery tube is be made from a fused silica such as quartz silica. In certain embodiments, the delivery tube is be made from stainless steel. In certain embodiments, the delivery tube has an inner diameter between 50-500 μm, between 100-450 μm, between 150-400 μm, between 200-350 μm, between 200-300 μm, of 200 μm, of 250 μm, or of 300 μm. In certain embodiments, the delivery tube has an inner diameter of 200 μm, 250 μm, 300 μm, or between 200-300 μm.

The delivery tube can include a delivery tip at the distal end of the delivery tube. The delivery tip can include one or more delivery tip openings. The delivery tip can be a blunt tip, a flat tip, a conic tip, a needle tip, a bevel tip or an angled tip. In certain embodiments, the delivery tip is fabricated or manufactured from material which may be used (i.e. can be identified and is safe to use) in magnetic resonance imaging (MRI).

In certain embodiments, the distal region of the cannula system includes a rigid tube element which has a proximal end and a distal end, and further includes a rigid tube channel extending through the rigid tube for the proximal rigid tube end to the distal rigid tube end. In certain embodiments, the rigid tube element has a length of 9.0-11.0 mm. In certain embodiments, the rigid tube element has a length of 10.0 mm. In certain embodiments, the rigid tube has an inner diameter of 750 μm, 800 μm, 850 μm, or between 750-850 μm. In certain embodiments, the rigid tube has an outer diameter of 2.0 mm, 2.5 mm, 3.0 μm, or between 2.0-3.0 mm. In certain embodiments, the rigid tube element is made from stainless steel, titanium, polyvinylchloride (PVC), polymethylmethacrylate (PMMA), or polytetrafluoroethylene (PTFE). In certain embodiments, the rigid tube element is made from a ceramic material, such as an alumina-based ceramic material or a zirconia-based ceramic material.

In certain embodiments, the delivery tube extends through the rigid tube channel of the rigid tube element and is longer than the rigid tube element such that a portion of the distal end of the delivery tube extends beyond the distal end of the rigid tube to form an exposed delivery tube element. In certain embodiments, the exposed delivery tube element of the delivery tube has a length of between 2.5-5.0 mm. In certain embodiments, the delivery tube has a length of 3.0-3.5 mm, 3.5-4.0 mm, 4.0-4.5 mm or 4.5-5.0 mm.

In certain embodiments, the distal region of the cannula system includes a capillary tube which has a capillary tube channel extending through the capillary tube from a distal end of the capillary tube to a proximal end of the capillary tube. In certain embodiments, the capillary tube extends through a rigid tube channel of a rigid tube and the delivery tube extends through the capillary tube channel. In certain embodiments, the capillary tube has an inner diameter which is sufficiently small to prevent liquid from flowing between the capillary tube and the delivery tube. In certain embodiments, the capillary tube has an outer diameter which is sufficiently large to prevent liquid from flowing between the capillary tube and the rigid tube element.

In certain embodiments, the cannula system includes a support tube which extends from the proximal region of the cannula system to the distal region of the cannula system, wherein the support tube includes a support tube channel extending through the support tube from a distal end of the support tube to a proximal end of the support tube, and wherein the delivery tube extends through the support tube channel from the proximal region of the cannula system to the distal region of the cannula system. In certain embodiments, the support tube is made from stainless steel, titanium, polyvinylchloride (PVC), and polymethylmethacrylate (PMMA), or polytetrafluoroethylene (PTFE).

In certain embodiments, the distal end of the support tube is adjacent to or in contact with the proximal end of the rigid tube element. In certain embodiments, a portion of the distal end of the rigid tube element extends into the support tube channel at the proximal end of the support tube. In certain embodiments, the cannula system includes a collar tube which extends over a portion of the distal end of the support tube and extends over a portion of the proximal end of the rigid tube element.

In certain embodiments, a cannula system of the present disclosure includes a cannula positioning guide (CPG). In certain embodiments, the CPG includes: a guide base which has an upper surface, a lower surface, and at least one edge surface; and at least one comb projection extending from the edge surface of the guide base. In certain embodiments, the guide base is suitable (e.g. shape, material, texture, conformation, etc) for connecting, appending or adhering the CPG directly or indirectly to a target delivery ara or target delivery tissue. In certain embodiments, the CPG is made from medical-grade materials, such as medical-grade materials suitable for use during magnetic resonance imaging (MRI).

In certain embodiments, the CPG includes a first pedicle and a second pedicle. In certain embodiments, both pedicles are positioned on the upper or lower surface of the guide base. In certain embodiments, the CPG includes an alignment fence juxtaposed between the first pedicle and the second pedicle. In certain embodiments, the CPG includes one or more clamps, clips or attachment devices for attaching a cannula tube to the CPG. In certain embodiments, the CPG includes on or more surgical accessories attached to the guide base, such as a battery-powered LED illumination device.

The present disclosure presents a kit which includes a cannula system of the present disclosure and an adhesive. In certain embodiments, the adhesive includes DuraSeal, DuraSeal Exact, Adherus, or Evicel.

The present disclosure presents a method of delivering a therapeutic agent, comprising: providing a cannula system of the present disclosure, and delivering a therapeutic agent through the delivery tube and out of the delivery tip of the cannula system. The therapeutic agent can be delivered to a target subject. In certain embodiments the therapeutic agent is delivered to parenchymal tissue of a target subject, including parenchymal tissue in the brain or the spine of a target subject. In certain embodiments, the therapeutic agent is delivered parenchymal tissue in the eye of a target subject. In certain embodiments, the therapeutic agent is delivered to the spine of a target subject, including a portion of the spinal parenchyma of a target subject or a non-parenchymal structure corresponding with a portion of the spinal parenchyma of a target subject. In certain embodiments, the spinal target is the posterior white column, the lateral white column, the anterior white column, the posterior gray horn, the lateral gray horn, the posterior gray commissure, the anterior gray commissure, the anterior white commissure, the central canal, the ventral horn, or a combination thereof. In certain embodiments, the therapeutic agent includes a small molecule compound, a naturally-occurring pharmaceutical compound, a synthetic pharmaceutical compound, a polypeptide, a protein, a polynucleotide, a viral vector, a biological progenitor cells, a stem cell, a biological transplant cell, a macrophage, a T cell, or a combination thereof. In certain embodiments, the therapeutic agent comprises an AAV viral vector. In certain embodiments, the therapeutic agent comprises an RNAi material, such as an siRNA material. In certain embodiments, the therapeutic agent comprises an AAV viral vector carrying an RNAi material such as an siRNA material. In certain embodiments, the RNAi material is siRNA. In certain embodiments, the RNAi material is a siRNA duplex targeting SOD1. In certain embodiments, the target subject has amyotrophic lateral sclerosis (ALS), or the therapeutic agent is used in treating amyotrophic lateral sclerosis (ALS).

In certain embodiments, the cannula system is adhered to the delivery target area using an adhesive. The adhesive can include one or more of DuraSeal, DuraSeal Exact, Adherus, and Evicel.

### BRIEF DESCRIPTION OF THE FIGURES

FIG. 1 presents a side view of cannula system 10, associated with certain embodiments of the present disclosure.

FIG. 2 presents a cross-sectional view of cannula system 10 taken along line 1-1 of FIG. 1.

FIG. 3 presents a cross-sectional view of the proximal section 12 of cannula system 10 as shown in FIG. 2.

FIG. 4 presents a cross-sectional view of a proximal section 112 of a cannula system, associated with certain embodiments of the present disclosure.

FIG. 5 presents a cross-sectional view of the distal section 14 of cannula system 10 as shown in FIG. 2.

FIG. 6 presents a cross-sectional view of a distal section 214 of a cannula system, associated with certain embodiments of the present disclosure.

FIG. 7A-7C present various embodiments of cannula delivery tips, associated with certain embodiments of the present disclosure.

FIG. 8 presents a front perspective view of cannula positioning guide 1100, associated with certain embodiments of the present disclosure.

FIG. 9 presents a top view of cannula positioning guide 1100 of FIG. 8.

FIG. 10 presents a top view of the cannula positioning guide 1100 as it may be utilized during a surgical procedure involving a laminectomy (components not shown to scale), associated with certain embodiments of the present disclosure.

FIG. 11 presents a cross-sectional view of spine 1300, illustrating cannula positioning guide 1100 as it may be utilized during a procedure involving a laminectomy (components not shown to scale), associated with certain embodiments of the present disclosure.

### DETAILED DESCRIPTION

### I. Cannula System

The present disclosure presents cannula systems for delivering a therapeutic agent to a target subject. In certain embodiments, the present disclosure presents a cannula system for delivering a therapeutic agent to parenchymal tissue of a target subject, such as tissue in the brain or the spine of the target subject.

In certain embodiments, the present disclosure presents a cannula system which includes a proximal section and a distal section.

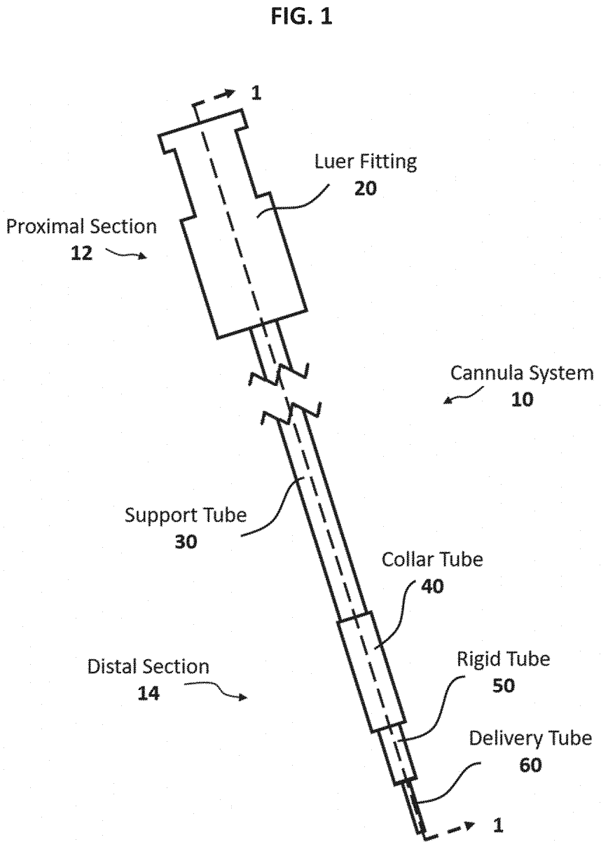

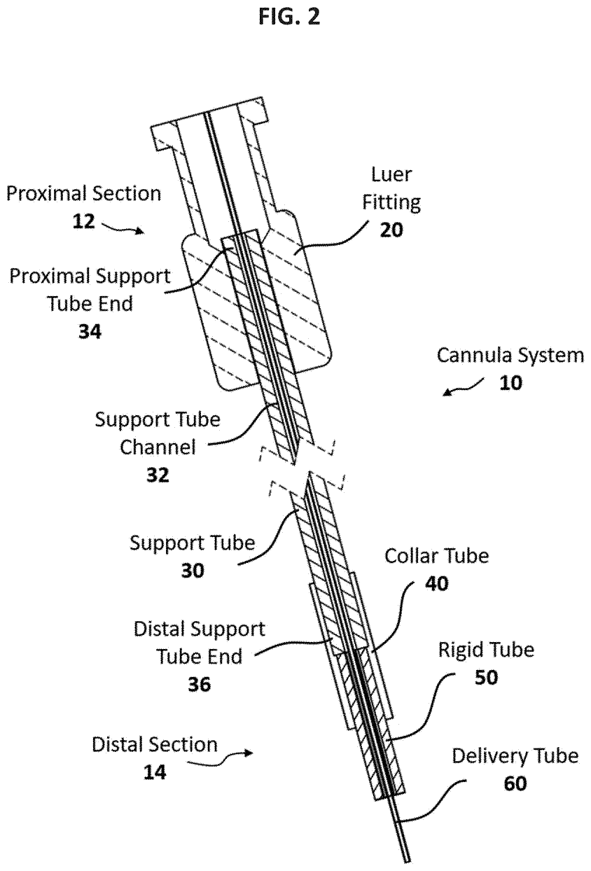

FIG. 1 and FIG. 2 are representative of one embodiment of a cannula system of the present disclosure. FIG. 1 presents a side view of cannula system 10. FIG. 2 presents a cross-sectional view of cannula system 10 taken along line 1-1 of FIG. 1. The cannula system 10 includes a proximal section 12 and a distal section 14. A support tube 30 extends from the proximal section 12 to the distal section 14. Support tube 30 includes a proximal support tube end 34 in the proximal section 12 of cannula system 10, and a distal support tube end 36 in the distal section 14 of cannula system 10. Within support tube 30 is a support tube channel 32, which extends from the proximal support tube end 34 to the distal support tube end 36 within support tube 30. Cannula system 10 also includes a delivery tube 60 which extends from the proximal section 12 to the distal section 14, passing through support tube channel 32 within support tube 30. The length of delivery tube 60 can be longer than the length of support tube 30, such that a proximal portion of delivery tube 60 extends beyond the proximal support tube end 34 of support tube 30 and a distal portion of delivery tube 60 extends beyond the distal support tube end 36 of support tube 30.

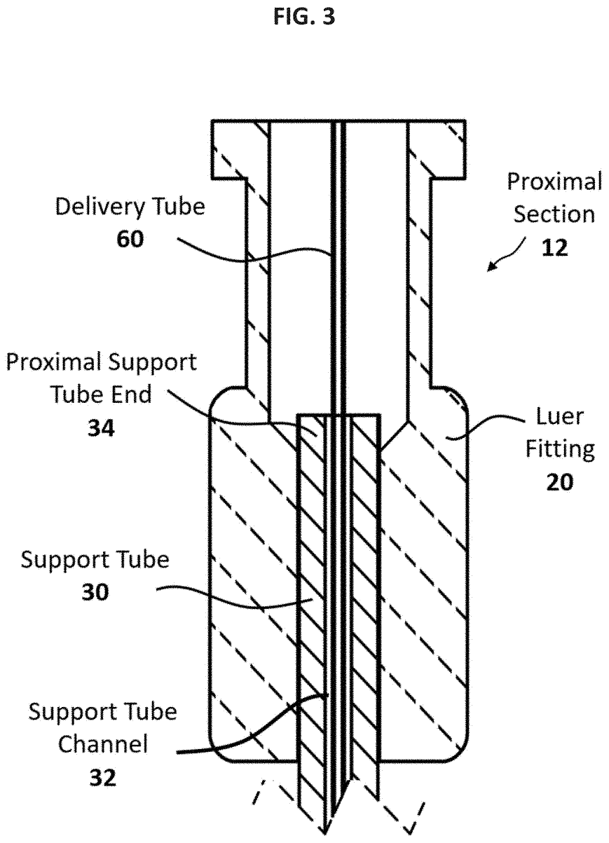

FIG. 3 presents a close-up view of the proximal section 12 of cannula system 10 as shown in FIG. 2. The proximal section 12 of cannula system 10 includes the proximal support tube end 34 of support tube 30, and a proximal portion of delivery tube 60. The length of delivery tube 60 is longer than the length of support tube 30, such that the proximal portion of delivery tube 60 extends beyond the proximal support tube end 34 of support tube 30. A luer fitting 20 is engaged with support tube 30, which allows the proximal section 12 of cannula system 10 to be operably coupled or engaged to other systems, such as pumps, extension tubes or guide systems. The luer adapter 20 can be bonded to the support tube 30 using an adhesive, such as a Loctite UV adhesive, or can be engaged with support tube 30 through frictional forces.

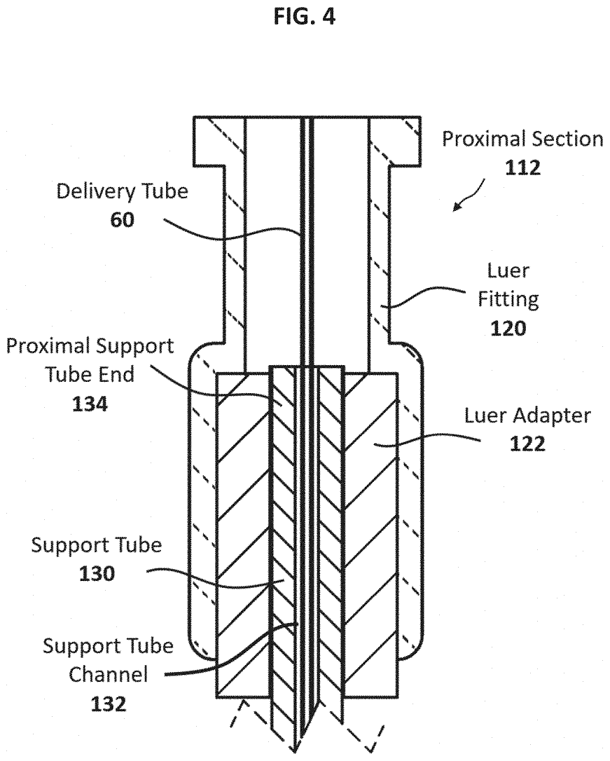

FIG. 4 presents a close-up, cross-sectional view of an embodiment of proximal section 112 of a cannula system. Proximal section 112 includes a proximal portion of support tube 130, which includes a proximal support tube end 134 and a proximal portion of support tube channel 132. Proximal section 112 also includes a luer adapter 122 which is engaged to the external surface of support tube 130, and also engaged to the internal surface of luer fitting 120. The diameter and thickness of luer adapter 122 can be adjusted to allow for proper engagement between support tube 130 and luer fitting 120.

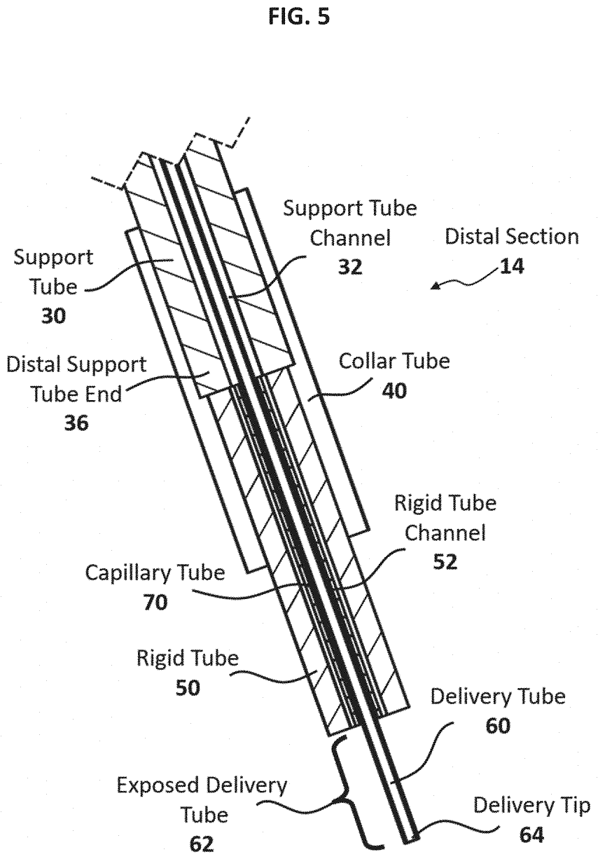

FIG. 5 presents a close-up view of the distal section 14 of cannula system 10 as shown in FIG. 2. The distal section 14 of cannula system 10 includes a rigid tube 50, which includes a rigid tube channel 52 which extends through the length of rigid tube 50. One end of rigid tube 50 is engaged with the distal support tube end 36 of support tube 30, such that rigid tube 50 is distally adjacent to support tube 30. Rigid tube 50 and support tube 30 can be arranged such that rigid tube channel 52 is co-axially aligned and operably engaged with support tube channel 32. The support tube 30 can be bonded to the rigid tube 50 using an adhesive, such as an epoxy or a Loctite UV adhesive. The junction of support tube 30 and rigid tube 50 can be enclosed with a collar tube 40, which extends over a portion of support tube 30 and over an adjacent portion of rigid tube 50. The collar tube 40 can be bonded to support tube 30 or to rigid tube 50 using an adhesive, such as an epoxy or a Loctite UV adhesive.

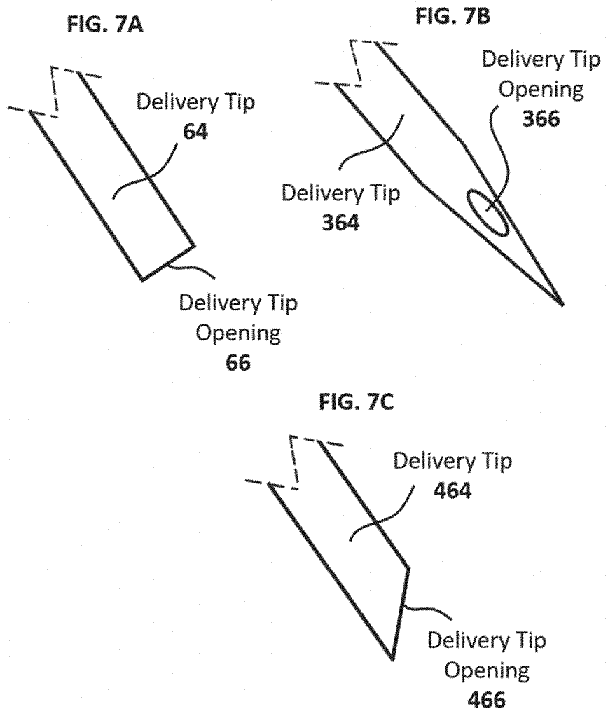

The distal section 14 of cannula system 10 further includes a distal portion of delivery tube 60 which extends through the support tube channel 32 of support tube 30 and through the rigid tube channel 52 of rigid tube 50. The length of delivery tube 60 is longer than the combined length of support tube 30 and rigid tube 50, such that the distal portion of delivery tube 60 extends beyond the rigid tube channel 52 of rigid tube 50, thus forming an exposed delivery tube 62. At the distal end of delivery tube 60 is a delivery tip 64, which includes a delivery opening 66 (see FIG. 7A) through which a fluid or therapeutic agent can be delivered. The distal section 14 of cannula system 10 can also further include a capillary tube 70 within rigid tube channel 52, wherein delivery tube 60 also extends through capillary tube 70.

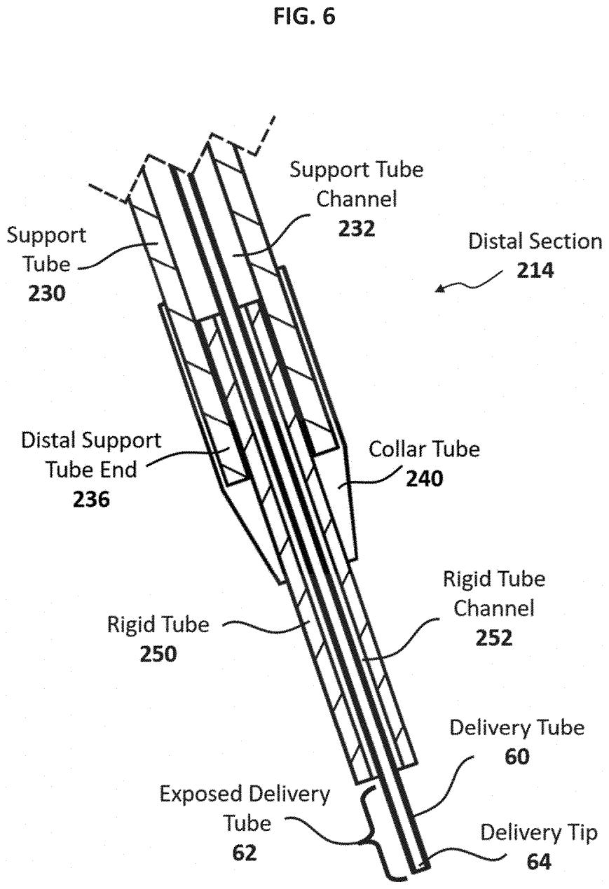

FIG. 6 presents a close-up, cross-sectional view of an embodiment of distal section 214 of a cannula system. Distal section 214 includes a distal portion of support tube 230, which includes a distal support tube end 236 and a distal portion of support tube channel 232. Distal section 214 also includes a rigid tube 250, which includes a rigid tube channel 252 which extends through the length of rigid tube 250. One end of rigid tube 250 is engaged with the distal support tube end 236 of support tube 230 by extending into a distal portion of support tube channel 232, as shown in FIG. 6. The support tube 230 can be bonded to the rigid tube 250 using an adhesive, such as a Loctite UV adhesive. The junction of support tube 230 and rigid tube 250 can be enclosed with a collar tube 240, which extends over a portion of support tube 230 and over an adjacent portion of rigid tube 250. The collar tube 240 can be bonded to support tube 230 or to rigid tube 250 using an adhesive, such as an epoxy or a Loctite UV adhesive.

FIG. 7A presents a close-up, perspective view of delivery tip 64 on delivery tube 60, including delivery opening 66 located on the blunt (flat) end of delivery tip 64. FIG. 7B presents a perspective view of delivery tip 364 on an embodiment of a delivery tube, including delivery opening 366 located on the conic needle tip of delivery tip 364. FIG. 7C presents a perspective view of delivery tip 464 on an embodiment of a delivery tube, including delivery opening 466 located on the bevel (angled) end of delivery tip 464.

### Delivery Tube

In certain embodiments, the cannula system includes a delivery tube (i.e. cannula tube) which extends from the proximal section to the distal section of the cannula system. The delivery tube can be made from one or more medical-grade materials including stainless steel, titanium, polyvinylchloride (PVC), polymethylmethacrylate (PMMA), polytetrafluoroethylene (PTFE) or a fused silica such as quartz silica. In certain embodiments, the delivery tube is made from a flexible material. In certain embodiments, the delivery tube includes a polymeric coating, such as a polyimide coating. In certain embodiments, the delivery tube has a diameter and thickness which allow the delivery tube to be flexible. In certain embodiments, the delivery tube is fabricated or manufactured from material which may be used (i.e. can be identified and is safe to use) in magnetic resonance imaging (MRI).

The delivery tube can have an inner diameter which facilitates effective delivery of a therapeutic agent to a target area. In certain embodiments, the delivery tube has an inner diameter of 50 μm, 100 μm, 150 μm, 200 μm, 250 μm, 300 μm, 350 μm, 400 μm, 450 μm, 500 μm, between 50-100 μm, between 50-150 μm, between 50-200 μm, between 50-250 μm, between 50-300 μm, between 50-350 μm, between 50-400 μm, between 50-450 μm, between 50-500 μm, between 100-150 μm, between 100-200 μm, between 100-250 μm, between 100-300 μm, between 100-350 μm, between 100-400 μm, between 100-450 μm, between 100-500 μm, between 150-200 μm, between 150-250 μm, between 150-300 μm, between 150-350 μm, between 150-400 μm, between 150-450 μm, between 150-500 μm, between 200-250 μm, between 200-300 μm, between 200-350 μm, between 200-400 μm, between 200-450 μm, between 200-500 μm, between 250-300 μm, between 250-350 μm, between 250-400 μm, between 250-450 μm, between 250-500 μm, between 300-350 μm, between 300-400 μm, between 300-450 μm, between 300-500 μm, between 350-400 μm, between 350-450 μm, between 350-500 μm, between 400-450 μm, between 400-500 μm, or between 450-500 μm. In certain embodiments, the delivery tube has an inner diameter of 150 μm. In certain embodiments, the delivery tube has an inner diameter of 200 μm. In certain embodiments, the delivery tube has an inner diameter of 250 μm. In certain embodiments, the delivery tube has an inner diameter of 300 μm. In certain embodiments, the delivery tube has an inner diameter between 200-300 μm.

The delivery tube can have a thickness which allows the delivery tube to be flexible. In certain embodiments, the delivery tube has a thickness of 50 μm, 100 μm, 150 μm, 200 μm, 250 μm, 300 μm, 350 μm, 400 μm, 450 μm, 500 μm, between 50-100 μm, between 50-150 μm, between 50-200 μm, between 50-250 μm, between 50-300 μm, between 50-350 μm, between 50-400 μm, between 50-450 μm, between 50-500 μm, between 100-150 μm, between 100-200 μm, between 100-250 μm, between 100-300 μm, between 100-350 μm, between 100-400 μm, between 100-450 μm, between 100-500 μm, between 150-200 μm, between 150-250 μm, between 150-300 μm, between 150-350 μm, between 150-400 μm, between 150-450 μm, between 150-500 μm, between 200-250 μm, between 200-300 μm, between 200-350 μm, between 200-400 μm, between 200-450 μm, between 200-500 μm, between 250-300 μm, between 250-350 μm, between 250-400 μm, between 250-450 μm, between 250-500 μm, between 300-350 μm, between 300-400 μm, between 300-450 μm, between 300-500 μm, between 350-400 μm, between 350-450 μm, between 350-500 μm, between 400-450 μm, between 400-500 μm, or between 450-500 μm. In certain embodiments, the delivery tube has a thickness of 50 μm. In certain embodiments, the delivery tube has a thickness of 100 μm. In certain embodiments, the delivery tube has a thickness of 150 μm. In certain embodiments, the delivery tube has a thickness of 200 μm. In certain embodiments, the delivery tube has a thickness between 50-150 μm.

The delivery tube includes a delivery tip at the distal end of the delivery tube. The delivery tip includes one or more delivery tip openings and can have any shape or configuration which facilitates effective delivery of a therapeutic agent to a target area through the delivery tip openings. In certain embodiments, the delivery tip is a blunt (flat) tip, a conic needle tip, or a bevel (angled) tip. In certain embodiments, the delivery tip is fabricated or manufactured from material which may be used (i.e. can be identified and is safe to use) in magnetic resonance imaging (MRI).

### Rigid Tube

In certain embodiments, a cannula system of the present disclosure includes a rigid tube. In certain embodiments, the rigid tube is located in the distal section of the cannula system. The rigid tube can be made from one or more medical-grade materials including stainless steel, titanium, polyvinylchloride (PVC), polymethylmethacrylate (PMMA), polytetrafluoroethylene (PTFE), or ceramics such as alumina-based ceramics and zirconia-based ceramics. In certain embodiments, the rigid tube is made from a rigid material. In certain embodiments, the rigid tube is fabricated or manufactured from material which may be used (i.e. can be identified and is safe to use) in magnetic resonance imaging (MRI).

In certain embodiments, the rigid tube can have a length of 5.0 mm, 6.0 mm, 7.0 mm, 8.0 mm, 9.0 mm, 10.0 mm, 11.0 mm, 12.0 mm, 13.0 mm, 14.0 mm, 15.0 mm, 16.0 mm, 17.0 mm, 18.0 mm, 19.0 mm, and 20.0 mm. The rigid tube can have a length between 1.0-20.0 mm, 2.0-19.0 mm, 3.0-18.0 mm, 4.0-17.0 mm, 5.0-16.0 mm, 5.0-15.0 mm, 6.0-15.0 mm, 7.0-15.0 mm, 8.0-20.0 mm, 9.0-20.0 mm, 10.0-20.0 mm, and 10.0-15.0 mm.

The rigid tube can include a rigid tube channel which extends through the length of the rigid tube from the proximal end of the rigid tube to the distal end of the rigid tube. The rigid tube can have an inner diameter which is larger than the outer diameter of a delivery tube, thereby allowing the delivery tube to extend through the rigid tube channel. In certain embodiments, the rigid tube has an inner diameter of 200 μm, 250 μm, 300 μm, 350 μm, 400 μm, 450 μm, 500 μm, 550 μm, 600 μm, 650 μm, 700 μm, between 200-250 μm, between 200-300 μm, between 200-350 μm, between 200-400 μm, between 200-450 μm, between 200-500 μm, between 200-550 μm, between 200-600 μm, between 200-650 μm, between 200-700 μm, between 250-300 μm, between 250-350 μm, between 250-400 μm, between 250-450 μm, between 250-500 μm, between 250-550 μm, between 250-600 μm, between 250-650 μm, between 250-700 μm, between 300-350 μm, between 300-400 μm, between 300-450 μm, between 300-500 μm, between 300-550 μm, between 300-600 μm, between 300-650 μm, between 300-700 μm, between 350-400 μm, between 350-450 μm, between 350-500 μm, between 350-550 μm, between 350-600 μm, between 350-650 μm, between 350-700 μm, between 400-450 μm, between 400-500 μm, between 400-550 μm, between 400-600 μm, between 400-650 μm, between 400-700 μm, between 450-500 μm, between 450-550 μm, between 450-600 μm, between 450-650 μm, between 450-700 μm, between 500-550 μm, between 500-600 μm, between 500-650 μm, between 500-700 μm, between 550-600 μm, between 550-650 μm, between 550-700 μm, between 600-650 μm, between 600-700 μm, between 650-700 μm. In certain embodiments, the rigid tube has an inner diameter of 350 μm. In certain embodiments, the rigid tube has an inner diameter of 400 μm. In certain embodiments, the rigid tube has an inner diameter of 450 μm. In certain embodiments, the rigid tube has an inner diameter of 500 μm. In certain embodiments, the rigid tube has an inner diameter of 550 μm. In certain embodiments, the rigid tube has an inner diameter of 600 μm. In certain embodiments, the rigid tube has an inner diameter between 350-550 μm.

The rigid tube can have a thickness which allows the delivery tube to be rigid. In certain embodiments, the delivery tube has a thickness of 50 μm, 100 μm, 150 μm, 200 μm, 250 μm, 300 μm, 350 μm, 400 μm, 450 μm, 500 μm, between 50-100 μm, between 50-150 μm, between 50-200 μm, between 50-250 μm, between 50-300 μm, between 50-350 μm, between 50-400 μm, between 50-450 μm, between 50-500 μm, between 100-150 μm, between 100-200 μm, between 100-250 μm, between 100-300 μm, between 100-350 μm, between 100-400 μm, between 100-450 μm, between 100-500 μm, between 150-200 μm, between 150-250 μm, between 150-300 μm, between 150-350 μm, between 150-400 μm, between 150-450 μm, between 150-500 μm, between 200-250 μm, between 200-300 μm, between 200-350 μm, between 200-400 μm, between 200-450 μm, between 200-500 μm, between 250-300 μm, between 250-350 μm, between 250-400 μm, between 250-450 μm, between 250-500 μm, between 300-350 μm, between 300-400 μm, between 300-450 μm, between 300-500 μm, between 350-400 μm, between 350-450 μm, between 350-500 μm, between 400-450 μm, between 400-500 μm, or between 450-500 μm. In certain embodiments, the rigid tube has a thickness of 150 μm. In certain embodiments, the rigid tube has a thickness of 200 μm. In certain embodiments, the rigid tube has a thickness of 250 μm. In certain embodiments, the rigid tube has a thickness of 300 μm. In certain embodiments, the rigid tube has a thickness between 150-300 μm.

In certain embodiments, the length of the delivery tube is longer than the length of the rigid tube, such that a distal portion of the delivery tube extends beyond the rigid tube channel of the rigid tube, thus forming an exposed delivery tube. As used herein, the term “exposed delivery tube” refers to a distal portion of a delivery tube which extends beyond a distal end of a rigid tube. The exposed delivery tube can have a length which facilitates the effective delivery of a therapeutic agent to a therapeutic target. In certain embodiments, the exposed delivery tube can have a length of 0.5 mm, 1.0 mm, 1.5 mm, 2.0 mm, 2.5 mm, 3.0 mm, 3.5 mm, 4.0 mm, 4.5 mm, 5.0 mm, 5.5 mm, 6.0 mm, 6.5 mm, 7.0 mm, 7.5 mm, 8.0 mm, 8.5 mm, 9.0 mm, 9.5 mm, 10.0 mm, between 0.5-1.0 mm, between 0.5-1.5 mm, between 0.5-2.0 mm, between 0.5-2.5 mm, between 0.5-3.0 mm, between 0.5-3.5 mm, between 0.5-4.0 mm, between 0.5-4.5 mm, between 0.5-5.0 mm, between 0.5-5.5 mm, between 0.5-6.0 mm, between 0.5-6.5 mm, between 0.5-7.0 mm, between 0.5-7.5 mm, between 0.5-8.0 mm, between 0.5-8.5 mm, between 0.5-9.0 mm, between 0.5-9.5 mm, between 0.5-10.0 mm, between 1.0-1.5 mm, between 1.0-2.0 mm, between 1.0-2.5 mm, between 1.0-3.0 mm, between 1.0-3.5 mm, between 1.0-4.0 mm, between 1.0-4.5 mm, between 1.0-5.0 mm, between 1.0-5.5 mm, between 1.0-6.0 mm, between 1.0-6.5 mm, between 1.0-7.0 mm, between 1.0-7.5 mm, between 1.0-8.0 mm, between 1.0-8.5 mm, between 1.0-9.0 mm, between 1.0-9.5 mm, between 1.0-10.0 mm, between 1.5-2.0 mm, between 1.5-2.5 mm, between 1.5-3.0 mm, between 1.5-3.5 mm, between 1.5-4.0 mm, between 1.5-4.5 mm, between 1.5-5.0 mm, between 1.5-5.5 mm, between 1.5-6.0 mm, between 1.5-6.5 mm, between 1.5-7.0 mm, between 1.5-7.5 mm, between 1.5-8.0 mm, between 1.5-8.5 mm, between 1.5-9.0 mm, between 1.5-9.5 mm, between 1.5-10.0 mm, between 2.0-2.5 mm, between 2.0-3.0 mm, between 2.0-3.5 mm, between 2.0-4.0 mm, between 2.0-4.5 mm, between 2.0-5.0 mm, between 2.0-5.5 mm, between 2.0-6.0 mm, between 2.0-6.5 mm, between 2.0-7.0 mm, between 2.0-7.5 mm, between 2.0-8.0 mm, between 2.0-8.5 mm, between 2.0-9.0 mm, between 2.0-9.5 mm, between 2.0-10.0 mm, between 2.5-3.0 mm, between 2.5-3.5 mm, between 2.5-4.0 mm, between 2.5-4.5 mm, between 2.5-5.0 mm, between 2.5-5.5 mm, between 2.5-6.0 mm, between 2.5-6.5 mm, between 2.5-7.0 mm, between 2.5-7.5 mm, between 2.5-8.0 mm, between 2.5-8.5 mm, between 2.5-9.0 mm, between 2.5-9.5 mm, between 2.5-10.0 mm, between 3.0-3.5 mm, between 3.0-4.0 mm, between 3.0-4.5 mm, between 3.0-5.0 mm, between 3.0-5.5 mm, between 3.0-6.0 mm, between 3.0-6.5 mm, between 3.0-7.0 mm, between 3.0-7.5 mm, between 3.0-8.0 mm, between 3.0-8.5 mm, between 3.0-9.0 mm, between 3.0-9.5 mm, between 3.0-10.0 mm, between 3.5-4.0 mm, between 3.5-4.5 mm, between 3.5-5.0 mm, between 3.5-5.5 mm, between 3.5-6.0 mm, between 3.5-6.5 mm, between 3.5-7.0 mm, between 3.5-7.5 mm, between 3.5-8.0 mm, between 3.5-8.5 mm, between 3.5-9.0 mm, between 3.5-9.5 mm, between 3.5-10.0 mm, between 4.0-4.5 mm, between 4.0-5.0 mm, between 4.0-5.5 mm, between 4.0-6.0 mm, between 4.0-6.5 mm, between 4.0-7.0 mm, between 4.0-7.5 mm, between 4.0-8.0 mm, between 4.0-8.5 mm, between 4.0-9.0 mm, between 4.0-9.5 mm, between 4.0-10.0 mm, between 4.5-5.0 mm, between 4.5-5.5 mm, between 4.5-6.0 mm, between 4.5-6.5 mm, between 4.5-7.0 mm, between 4.5-7.5 mm, between 4.5-8.0 mm, between 4.5-8.5 mm, between 4.5-9.0 mm, between 4.5-9.5 mm, between 4.5-10.0 mm, between 5.0-5.5 mm, between 5.0-6.0 mm, between 5.0-6.5 mm, between 5.0-7.0 mm, between 5.0-7.5 mm, between 5.0-8.0 mm, between 5.0-8.5 mm, between 5.0-9.0 mm, between 5.0-9.5 mm, between 5.0-10.0 mm, between 5.5-6.0 mm, between 5.5-6.5 mm, between 5.5-7.0 mm, between 5.5-7.5 mm, between 5.5-8.0 mm, between 5.5-8.5 mm, between 5.5-9.0 mm, between 5.5-9.5 mm, between 5.5-10.0 mm, between 6.0-6.5 mm, between 6.0-7.0 mm, between 6.0-7.5 mm, between 6.0-8.0 mm, between 6.0-8.5 mm, between 6.0-9.0 mm, between 6.0-9.5 mm, between 6.0-10.0 mm, 6.5-7.0 mm, between 6.5-7.5 mm, between 6.5-8.0 mm, between 6.5-8.5 mm, between 6.5-9.0 mm, between 6.5-9.5 mm, between 6.5-10.0 mm, between 7.0-7.5 mm, between 7.0-8.0 mm, between 7.0-8.5 mm, between 7.0-9.0 mm, between 7.0-9.5 mm, between 7.0-10.0 mm, between 7.5-8.0 mm, between 7.5-8.5 mm, between 7.5-9.0 mm, between 7.5-9.5 mm, between 7.5-10.0 mm, between 8.0-8.5 mm, between 8.0-9.0 mm, between 8.0-9.5 mm, between 8.0-10.0 mm, between 8.5-9.0 mm, between 8.5-9.5 mm, between 8.5-10.0 mm, between 9.0-9.5 mm, and between 9.0-10.0 mm. In certain embodiments, the exposed delivery tube has a length of 2.5 mm. In certain embodiments, the exposed delivery tube has a length of 3.0 mm. In certain embodiments, the exposed delivery tube has a length of 3.5 mm. In certain embodiments, the exposed delivery tube has a length of 4.0 mm. In certain embodiments, the exposed delivery tube has a length of 4.5 mm. In certain embodiments, the exposed delivery tube has a length of 5.0 mm. In certain embodiments, the exposed delivery tube has a length of 5.5 mm. In certain embodiments, the exposed delivery tube has a length between 3.0-5.0 mm. In certain embodiments, the exposed delivery tube has a length between 3.0-4.0 mm. In certain embodiments, the exposed delivery tube has a length between 4.0-5.0 mm.

### Capillary and Support Tubes

In certain embodiments, the distal section of the cannula system can include a capillary tube which extends through the rigid tube channel of the rigid tube. The capillary tube can include a capillary tube channel, with the delivery tube extending through the capillary tube channel such that the capillary tube is located between the outer surface of the delivery tube and the inner surface of the rigid tube. In certain embodiments, the capillary is the same length as the rigid tube. In certain embodiments, the capillary tube is shorter than the rigid tube, such that the entire length of the capillary tube can be contained with the rigid tube. In certain embodiments, the capillary tube has an inner diameter which allows the delivery tube to extend through the capillary tube channel, but which also prevents fluids to flow between the capillary tube and the delivery tube. In certain embodiments, the capillary tube has an outer diameter which allows the capillary tube to extend through the rigid tube channel, but which also prevents fluids to flow between the capillary tube and the rigid tube. In certain embodiments, the capillary tube is fabricated or manufactured from material which may be used (i.e. can be identified and is safe to use) in magnetic resonance imaging (MRI).

In certain embodiments, a cannula system of the present disclosure can include a support tube. The support tube can extend from the proximal section to the distal section of the cannula system. The support tube can be made from one or more medical-grade materials including stainless steel, titanium, polyvinylchloride (PVC), polymethylmethacrylate (PMMA), polytetrafluoroethylene (PTFE) or a fused silica such as quartz silica. In certain embodiments, the support tube is made from a flexible material. In certain embodiments, the support tube has a diameter and thickness which allow the delivery tube to be flexible. In certain embodiments, the support tube is fabricated or manufactured from material which may be used (i.e. can be identified and is safe to use) in magnetic resonance imaging (MRI).

The support tube can include a support tube channel which extends through the length of the support tube from the proximal end of the support tube to the distal end of the support tube. The support tube can have an inner diameter which is larger than the outer diameter of a delivery tube, thereby allowing the delivery tube to extend through the support tube channel. In certain embodiments, the support tube has an inner diameter of 200 μm, 250 μm, 300 μm, 350 μm, 400 μm, 450 μm, 500 μm, 550 μm, 600 μm, 650 μm, 700 μm, between 200-250 μm, between 200-300 μm, between 200-350 μm, between 200-400 μm, between 200-450 μm, between 200-500 μm, between 200-550 μm, between 200-600 μm, between 200-650 μm, between 200-700 μm, between 250-300 μm, between 250-350 μm, between 250-400 μm, between 250-450 μm, between 250-500 μm, between 250-550 μm, between 250-600 μm, between 250-650 μm, between 250-700 μm, between 300-350 μm, between 300-400 μm, between 300-450 μm, between 300-500 μm, between 300-550 μm, between 300-600 μm, between 300-650 μm, between 300-700 μm, between 350-400 μm, between 350-450 μm, between 350-500 μm, between 350-550 μm, between 350-600 μm, between 350-650 μm, between 350-700 μm, between 400-450 μm, between 400-500 μm, between 400-550 μm, between 400-600 μm, between 400-650 μm, between 400-700 μm, between 450-500 μm, between 450-550 μm, between 450-600 μm, between 450-650 μm, between 450-700 μm, between 500-550 μm, between 500-600 μm, between 500-650 μm, between 500-700 μm, between 550-600 μm, between 550-650 μm, between 550-700 μm, between 600-650 μm, between 600-700 μm, between 650-700 μm. In certain embodiments, the support tube has an inner diameter of 350 μm. In certain embodiments, the support tube has an inner diameter of 400 μm. In certain embodiments, the support tube has an inner diameter of 450 μm. In certain embodiments, the support tube has an inner diameter of 500 μm. In certain embodiments, the support tube has an inner diameter of 550 μm. In certain embodiments, the support tube has an inner diameter of 600 μm. In certain embodiments, the support tube has an inner diameter between 350-550 μm.

The support tube can have a thickness which allows the support tube to be flexible. In certain embodiments, the support tube has a thickness of 50 μm, 100 μm, 150 μm, 200 μm, 250 μm, 300 μm, 350 μm, 400 μm, 450 μm, 500 μm, between 50-100 μm, between 50-150 μm, between 50-200 μm, between 50-250 μm, between 50-300 μm, between 50-350 μm, between 50-400 μm, between 50-450 μm, between 50-500 μm, between 100-150 μm, between 100-200 μm, between 100-250 μm, between 100-300 μm, between 100-350 μm, between 100-400 μm, between 100-450 μm, between 100-500 μm, between 150-200 μm, between 150-250 μm, between 150-300 μm, between 150-350 μm, between 150-400 μm, between 150-450 μm, between 150-500 μm, between 200-250 μm, between 200-300 μm, between 200-350 μm, between 200-400 μm, between 200-450 μm, between 200-500 μm, between 250-300 μm, between 250-350 μm, between 250-400 μm, between 250-450 μm, between 250-500 μm, between 300-350 μm, between 300-400 μm, between 300-450 μm, between 300-500 μm, between 350-400 μm, between 350-450 μm, between 350-500 μm, between 400-450 μm, between 400-500 μm, or between 450-500 μm. In certain embodiments, the support tube has a thickness of 150 μm. In certain embodiments, the support tube has a thickness of 200 μm. In certain embodiments, the support tube has a thickness of 250 μm. In certain embodiments, the support tube has a thickness of 300 μm. In certain embodiments, the support tube has a thickness between 150-300 μm.

In certain embodiments, the support tube can include a proximal end in the proximal section of the cannula system and a distal end in the distal section of the cannula system. In certain embodiments, the length of a delivery tube is greater than the length of the support tube. In certain embodiments, the proximal end of the delivery tube extends beyond the proximal end of the support tube. In certain embodiments, the distal end of the delivery tube extends beyond the distal end of the support tube.

In certain embodiments, the distal end of the support tube is in contact with or adjacent to the proximal end of a rigid tube element. The distal end of the support tube can be bonded to the proximal end of the rigid tube using an adhesive, such as an epoxy or a Loctite UV adhesive.

In certain embodiments, the junction of the support tube the and rigid tube can be enclosed with a collar tube. The collar tube can extend over a portion of support tube and over an adjacent portion of rigid tube. The collar tube can be bonded to support tube or to the rigid tube using an adhesive, such as an epoxy or a Loctite UV adhesive.

In certain embodiment, the support tube has an inner diameter which allows a portion of the rigid tube to extend into a portion of the support tube channel within the support tube.

### Cannula Positioning Guide (CPG)

The present disclosure presents a cannula system which includes a cannula positioning guide (CPG). The cannula positioning guide can be made from one or more medical-grade materials including stainless steel, titanium, polyvinylchloride (PVC), polymethylmethacrylate (PMMA), polytetrafluoroethylene (PTFE), any polymer or copolymer or ceramics such as alumina-based ceramics and zirconia-based ceramics. In certain embodiments, the cannula positioning guide is made from a rigid material. In certain embodiments, the cannula positioning guide is made from a pliable or flexible material. In certain embodiments, components of the cannula positioning guide, e.g., base, comb projections, pedicles and/or the alignment fence are independently made from different materials. In certain embodiments, the CPG is fabricated or manufactured from material which may be used in magnetic resonance imaging (MRI).

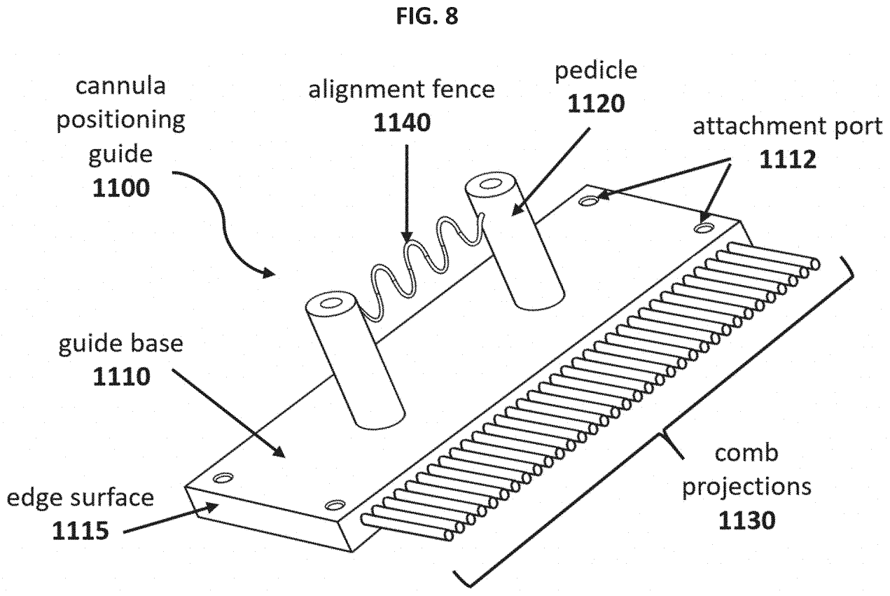

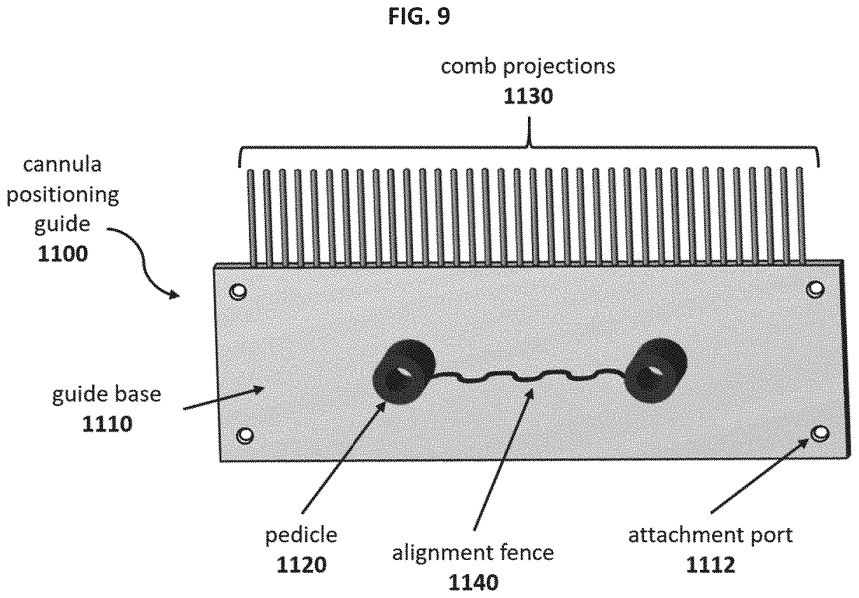

FIG. 8 and FIG. 9 are representative of certain embodiments of a cannula positioning guide of the present disclosure. FIG. 8 presents a front perspective view of the cannula positioning guide (CPG) 100. FIG. 9 presents a top view of the cannula positioning guide (CPG) 100.

The CPG includes a guide base 1110, and a plurality of comb projections 1130. The comb projections may extend from a single edge surface 1115 of the guide base 1110 or from multiple edge surfaces. The comb projections may vary independently in number, size, length, diameter, shape, volume and material. They may be less than 1 mm, 1 mm-5 mm, 1 mm-10 mm, 1 mm-20 mm, 1 mm, 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.6 mm, 2.7 mm, 2.8 mm, 2.9 mm, 3 mm, 3.1 mm, 3.2 mm, 3.3 mm, 3.4 mm, 3.5 mm, 3.6 mm, 3.7 mm, 3.8 mm, 3.9 mm, 4 mm, 4.1 mm, 4.2 mm, 4.3 mm, 4.4 mm, 4.5 mm, 4.6 mm, 4.7 mm, 4.8 mm, 4.9 mm, 5 mm, 5.1 mm, 5.2 mm, 5.3 mm, 5.4 mm, 5.5 mm, 5.6 mm, 5.7 mm, 5.8 mm, 5.9 mm, 6 mm, 6.1 mm, 6.2 mm, 6.3 mm, 6.4 mm, 6.5 mm, 6.6 mm, 6.7 mm, 6.8 mm, 6.9 mm, 7 mm, 7.1 mm, 7.2 mm, 7.3 mm, 7.4 mm, 7.5 mm, 7.6 mm, 7.7 mm, 7.8 mm, 7.9 mm, 8 mm, 8.1 mm, 8.2 mm, 8.3 mm, 8.4 mm, 8.5 mm, 8.6 mm, 8.7 mm, 8.8 mm, 8.9 mm, 9 mm, 9.1 mm, 9.2 mm, 9.3 mm, 9.4 mm, 9.5 mm, 9.6 mm, 9.7 mm, 9.8 mm, 9.9 mm, 10 mm, 1 mm-1 cm, 1 cm, 1.1 cm, 1.2 cm, 1.3 cm, 1.4 cm, 1.5 cm, 1.6 cm, 1.7 cm, 1.8 cm, 1.9 cm, 2 cm, 2.1 cm, 2.2 cm, 2.3 cm, 2.4 cm, 2.5 cm, 2.6 cm, 2.7 cm, 2.8 cm, 2.9 cm, 3 cm, or more in length or diameter.

The number of comb projections may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more.

The comb projections may be spaced evenly or in groups of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more. The comb projections may be centered on an edge surface or positioned on one or more longitudinal ends. They may be randomly arranged or arranged in patterns.

The base may further contain one or more attachment ports 1112 or passages useful in attaching the base to a tissue or biological substrate.

The attachment ports may pass through the entirety of the base or may not pass through the entirety of the base. The attachment ports may be on one or both surfaces of the base. The lumen of the attachment ports may be threaded, grooved or smooth and may be threaded, grooved or smooth on one or both ends. The diameter of any part or the whole of the port lumen may be less than 1 mm, 1 mm-5 mm, 1 mm-10 mm, 1 mm-20 mm, 1 mm, 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.6 mm, 2.7 mm, 2.8 mm, 2.9 mm, 3 mm, 3.1 mm, 3.2 mm, 3.3 mm, 3.4 mm, 3.5 mm, 3.6 mm, 3.7 mm, 3.8 mm, 3.9 mm, 4 mm, 4.1 mm, 4.2 mm, 4.3 mm, 4.4 mm, 4.5 mm, 4.6 mm, 4.7 mm, 4.8 mm, 4.9 mm, 5 mm, 5.1 mm, 5.2 mm, 5.3 mm, 5.4 mm, 5.5 mm, 5.6 mm, 5.7 mm, 5.8 mm, 5.9 mm, 6 mm, 6.1 mm, 6.2 mm, 6.3 mm, 6.4 mm, 6.5 mm, 6.6 mm, 6.7 mm, 6.8 mm, 6.9 mm, 7 mm, 7.1 mm, 7.2 mm, 7.3 mm, 7.4 mm, 7.5 mm, 7.6 mm, 7.7 mm, 7.8 mm, 7.9 mm, 8 mm, 8.1 mm, 8.2 mm, 8.3 mm, 8.4 mm, 8.5 mm, 8.6 mm, 8.7 mm, 8.8 mm, 8.9 mm, 9 mm, 9.1 mm, 9.2 mm, 9.3 mm, 9.4 mm, 9.5 mm, 9.6 mm, 9.7 mm, 9.8 mm, 9.9 mm, 10 mm, 1 mm-1 cm, 1 cm, 1.1 cm, 1.2 cm, 1.3 cm, 1.4 cm, 1.5 cm, 1.6 cm, 1.7 cm, 1.8 cm, 1.9 cm, 2 cm, 2.1 cm, 2.2 cm, 2.3 cm, 2.4 cm, 2.5 cm, 2.6 cm, 2.7 cm, 2.8 cm, 2.9 cm, 3 cm, or more.

In some embodiments, the CPG contains one or more pedicles 1120 on the upper or lower surface of the guide base 1110 or on both surfaces. The pedicles 1120 may be solid or hollow. They may contain a lumen which extends through the length of the pedicle or which extends only partially through the pedicle.

The pedicles may be of any length. For example, pedicles may be less than 1 mm, 1 mm-5 mm, 1 mm-10 mm, 1 mm-20 mm, 1 mm, 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.6 mm, 2.7 mm, 2.8 mm, 2.9 mm, 3 mm, 3.1 mm, 3.2 mm, 3.3 mm, 3.4 mm, 3.5 mm, 3.6 mm, 3.7 mm, 3.8 mm, 3.9 mm, 4 mm, 4.1 mm, 4.2 mm, 4.3 mm, 4.4 mm, 4.5 mm, 4.6 mm, 4.7 mm, 4.8 mm, 4.9 mm, 5 mm, 5.1 mm, 5.2 mm, 5.3 mm, 5.4 mm, 5.5 mm, 5.6 mm, 5.7 mm, 5.8 mm, 5.9 mm, 6 mm, 6.1 mm, 6.2 mm, 6.3 mm, 6.4 mm, 6.5 mm, 6.6 mm, 6.7 mm, 6.8 mm, 6.9 mm, 7 mm, 7.1 mm, 7.2 mm, 7.3 mm, 7.4 mm, 7.5 mm, 7.6 mm, 7.7 mm, 7.8 mm, 7.9 mm, 8 mm, 8.1 mm, 8.2 mm, 8.3 mm, 8.4 mm, 8.5 mm, 8.6 mm, 8.7 mm, 8.8 mm, 8.9 mm, 9 mm, 9.1 mm, 9.2 mm, 9.3 mm, 9.4 mm, 9.5 mm, 9.6 mm, 9.7 mm, 9.8 mm, 9.9 mm, 10 mm, 1 mm-1 cm, 1 cm, 1.1 cm, 1.2 cm, 1.3 cm, 1.4 cm, 1.5 cm, 1.6 cm, 1.7 cm, 1.8 cm, 1.9 cm, 2 cm, 2.1 cm, 2.2 cm, 2.3 cm, 2.4 cm, 2.5 cm, 2.6 cm, 2.7 cm, 2.8 cm, 2.9 cm, 3 cm, or more.

The lumen of the pedicle, when present, may be threaded, grooved or smooth and may be threaded, grooved or smooth on one or both ends. The diameter of any part or the whole of the lumen may be less than 1 mm, 1 mm-5 mm, 1 mm-10 mm, 1 mm-20 mm, 1 mm, 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.6 mm, 2.7 mm, 2.8 mm, 2.9 mm, 3 mm, 3.1 mm, 3.2 mm, 3.3 mm, 3.4 mm, 3.5 mm, 3.6 mm, 3.7 mm, 3.8 mm, 3.9 mm, 4 mm, 4.1 mm, 4.2 mm, 4.3 mm, 4.4 mm, 4.5 mm, 4.6 mm, 4.7 mm, 4.8 mm, 4.9 mm, 5 mm, 5.1 mm, 5.2 mm, 5.3 mm, 5.4 mm, 5.5 mm, 5.6 mm, 5.7 mm, 5.8 mm, 5.9 mm, 6 mm, 6.1 mm, 6.2 mm, 6.3 mm, 6.4 mm, 6.5 mm, 6.6 mm, 6.7 mm, 6.8 mm, 6.9 mm, 7 mm, 7.1 mm, 7.2 mm, 7.3 mm, 7.4 mm, 7.5 mm, 7.6 mm, 7.7 mm, 7.8 mm, 7.9 mm, 8 mm, 8.1 mm, 8.2 mm, 8.3 mm, 8.4 mm, 8.5 mm, 8.6 mm, 8.7 mm, 8.8 mm, 8.9 mm, 9 mm, 9.1 mm, 9.2 mm, 9.3 mm, 9.4 mm, 9.5 mm, 9.6 mm, 9.7 mm, 9.8 mm, 9.9 mm, 10 mm, 1 mm-1 cm, 1 cm, 1.1 cm, 1.2 cm, 1.3 cm, 1.4 cm, 1.5 cm, 1.6 cm, 1.7 cm, 1.8 cm, 1.9 cm, 2 cm, 2.1 cm, 2.2 cm, 2.3 cm, 2.4 cm, 2.5 cm, 2.6 cm, 2.7 cm, 2.8 cm, 2.9 cm, 3 cm, or more.

In some embodiments, the pedicles are fabricated with the base. In some embodiments, the pedicles are fabricated separately and attached to the base. Attachment may be by a weld, an adhesive, a screw, a linker or any other means of attachment.

The pedicles may be of any shape. The pedicles may be of any shape with a lumen having any shape. In some embodiments, the lumen may be concentric with the shape of the pedicle.

In some embodiments, an alignment fence 1140 may be juxtaposed between two pedicles 1120. In some embodiments, the alignment fence may serve as a cannula support, a stabilization guide, a clamp or clip substrate, a cannula or surgical instrument guide. The alignment fence may be made of any material suitable for the surgical suite or a medical procedure. The alignment fence may be straight, curved, irregular, undulating, arched, bowed or other shape. The alignment fence may comprise one or a plurality of components, wires, strings, bands, strips, rods, links or other connections between pedicles.

In some embodiments the base 1110 of the CPG may have attached or may incorporate one or more signaling devices. Such signaling devices may be a leveling mechanism, temperature probe, pH meter or other detector suitable for use in the surgical field or medical procedure. The base may be fabricated to contain one or more trajectory guides.

In some embodiments the base may be square, rectangle, irregular, circular in overall shape. The base may be of any size suitable to the application. The base may be planar (flat) or have any degree of curvature. The base may be concave or convex. The base may form a wedge or be angled in multiple directions from center along the long or short axis. The base may contain any number of ports or posts.

The CPG or any of its components may have one or more layers or coatings. Such coatings may serve to assist or resist glidance, to shield any part to which it is applied, and/or to be used as a delivery depot for any therapeutic or inert ingredient, drug or material. Layers or coatings may be manufactured and applied using standard techniques known in the art.

### II. Use of a Cannula System

The present disclosure presents cannula systems for delivering a therapeutic agent to a target subject. The present disclosure presents methods of using a cannula system for delivering a therapeutic agent to a target subject. In certain embodiments, the method includes delivering a therapeutic agent to parenchymal tissue of a target subject, such as tissue in the brain or the spine of a target subject.

In certain embodiments, the methods of using a cannula system includes one or more surgical procedures to provide access to the therapeutic target. Surgical procedures can include minimally invasive procedures, such as the use of a laminectomy to provide access to the spinal cord of a target subject. As a non-limiting example, a laminectomy can be performed by making a small incision over the target spinal area and displacing any muscles or soft tissue, thereby exposing the vertebral bones above the therapeutic target. An appropriate section of the vertebral bone can be removed (using a bone saw or other cutting tool) to exposes the dorsal surface of the spinal target.

Once the therapeutic target tissue is accessible (surgically or naturally), a cannula system is provided for delivering the therapeutic agent to the therapeutic target tissue. The proximal section of the cannula system can be fixed to the target subject at a location near the therapeutic target tissue to stabilize the cannula system during delivery. The cannula system should be arranged such that the delivery tip of the delivery tube can be functionally inserted into the delivery target area (i.e. target tissue). The delivery target area can be the therapeutic target, an ara adjacent to the therapeutic target, or an area (such as the heart) which facilitates the delivery of the therapeutic agent to the therapeutic target. The cannula system can be arranged using surgical targeting equipment or can be arranged free hand.

In certain embodiments, a cannula positioning guide (CPG) may be used to stabilize, align or otherwise be employed to adapt the trajectory of the cannula to the desired location for delivery of the therapeutic agent. The CPG of the cannula system can be fixed to the subject at a location near the target tissue. This can stabilize the cannula as the therapeutic agent is delivered into the target tissue. In certain embodiments, the cannula is attached to the CPG using a clip or clasp mechanism. In certain embodiments, the cannula system (e.g. cannula or CPG) is adhered to the delivery target area using an adhesive. The adhesive can include one or more of DuraSeal, DuraSeal Exact, Adherus, and Evicel.

Once the cannula system is position and the cannula is arranged for functional insertion into the delivery target area, a portion of the cannula can be inserted into the delivery target. In certain embodiments, the delivery tube is inserted into the delivery target up to the distal end of the rigid tube, such that the exposed delivery tube is the only section of the cannula system inserted into the delivery target. In certain embodiments, the delivery tube is inserted into the delivery target up to the distal end of the collar tube, such that the exposed delivery tube and a distal portion of the rigid tube are the only sections of the cannula system inserted into the delivery target. A therapeutically effective amount of the therapeutic agent can then be delivered to the therapeutic target tissue by injecting the therapeutic agent through the cannula system and into the delivery target area. Therapeutic agents can be delivered to the target in burst or bolus dosages, or delivered as part of a controlled or sustained release dosage for calculated release into a spinal cord target. In certain embodiments, the cannula system (e.g. CPG of the cannula system) can be secured to stabilizing structure or delivery platform to allow for long-duration delivery of the therapeutic agent to the therapeutic target tissue. Once the delivery of the therapeutic agent is completed, the cannula system can be withdrawn from the target delivery area and removed from the target patient. Surgical procedures can then be used to repair damaged tissue and complete the treatment procedure.

In certain embodiments, the delivery tube and/or delivery tip is fabricated or manufactured from material which may be used (i.e. can be identified and is safe to use) in magnetic resonance imaging (MRI). In certain embodiments, the surgical procedure includes the use of real-time MRI to follow the placement of the cannula system and the delivery of the therapeutic agent through the delivery tip of the cannula.

In certain embodiments, a cannula system of the present disclosure can be used in delivering RNAi materials, in delivering AAV compounds, or in methods of treating amyotrophic lateral sclerosis (ALS) as disclosed in PCT Patent Publication WO 2016077687 or WO 2016077689, the contents each of which are incorporated by reference herein in their entirety.

The present disclosure presents cannula systems having a cannula positioning guide (CPG) and methods for delivering a therapeutic agent to a subject using such systems. In certain embodiments, the method includes delivering a therapeutic agent to tissue of a subject, such as a target tissue in the CNS, brain, spine or spinal cord of a subject via a cannula which is aligned or positioned using one or more CPGs. In certain embodiments, more than one cannula may engage with a cannula positioning guide.

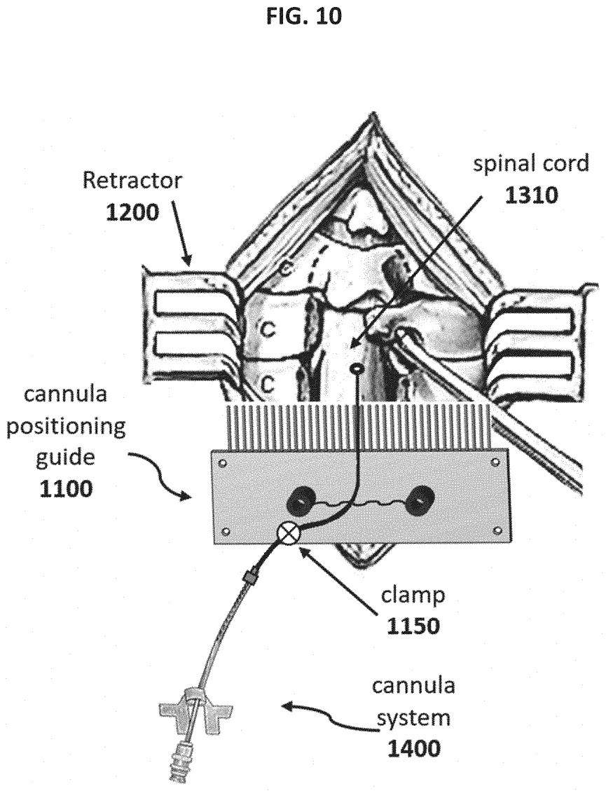

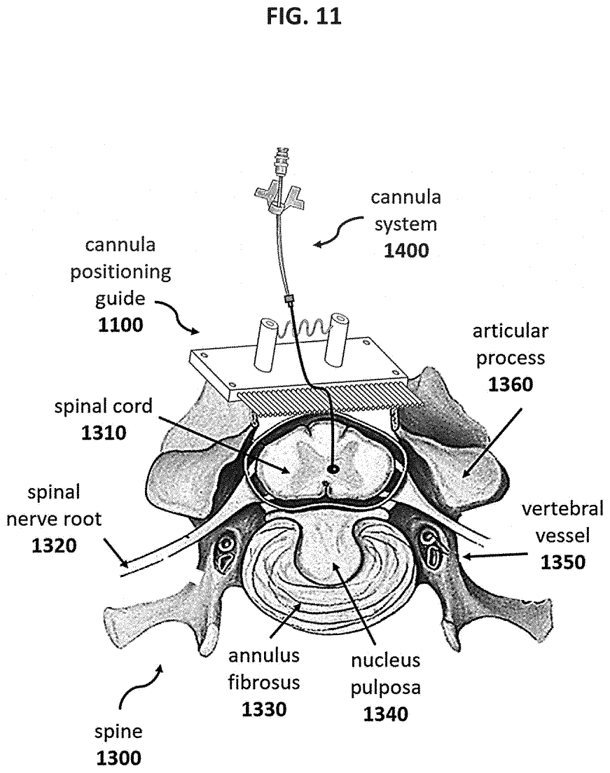

FIG. 10 presents a top view of the cannula positioning guide 1100 as it may be utilized during a surgical procedure involving a laminectomy (components not shown to scale). FIG. 11 presents a cross-sectional view of spine 1300, illustrating cannula positioning guide 1100 as it may be utilized during a procedure involving a laminectomy (components not shown to scale). Components of spine 1300 are shown, including spinal cord 1310, spinal nerve root 1320, annulus fibrosus 1330, nucleus pulposa 1340, vertebra vessel 1350, and articular process 1360.

A laminectomy is performed by making a small incision over the target spinal cord 1310 and displacing any muscles or soft tissue using retractor 1200, thereby exposing the vertebral bones above the therapeutic target. An appropriate section of the vertebral bone is be removed (using a bone saw or other cutting tool) to exposes the dorsal surface of spinal cord 1310. The cannula positioning guide (CPG) 1100 of cannula system 1400 is fixed to the subject using an adhesive at a location near the target tissue of spinal cord 1310. The cannula of cannula system 1400 is attached to the CPG 1100 using a clip or clamp mechanism 1150.

The cannula is arranged for functional insertion into the target area of spinal cord 1310 using the alignment fence of CPG 1100, such that the exposed delivery tube of the cannula can be inserted into the delivery target. A therapeutically effective amount of the therapeutic agent is then be delivered to the therapeutic target tissue by injecting the therapeutic agent through the cannula system and into the delivery target area. Once the delivery of the therapeutic agent is completed, the cannula system is withdrawn from the target delivery area and surgical procedures are be used to repair damaged tissue and complete the treatment procedure.

### III. Compositions for Delivery

### Therapeutic Agents

The present disclosure presents methods of administering an therapeutically effective amount of a therapeutic agent to a therapeutic target tissue in a subject.

The therapeutic target tissue can be selected based on several factors, including (but not limited to): the type of ailment, disease or disorder being treated; the anatomy and physiology of the target subject and the target treatment area, the accessibility of the target treatment area; and the nature of the therapeutic agent being administered.

In certain embodiment, the therapeutic target (i.e. target tissue) is in the spine of a target subject, such as the spinal cord. The spinal target can comprise any portion of the spinal parenchyma or corresponding non-parenchymal structures. Therapeutic target tissue in the spine can include (but are not limited to): the posterior white column, the lateral white column, the anterior white column, the posterior gray horn, the lateral gray horn, the posterior gray commissure, the anterior gray commissure, the anterior white commissure, the central canal, the ventral horn, and combinations thereof.

Non-limiting examples of therapeutic agents can include (but are not limited to): small molecules and pharmaceutical compounds (naturally-occurring or synthetic), polypeptides (such as proteins), polynucleotides, viral vectors, biological progenitor cells (such as stem cells), and biological transplant cells (such as macrophages and T cells).

The amount of therapeutic agent delivered to the therapeutic target tissue will depend on the transduction and potency properties of the agent, as well as the type and severity of the ailment, disorder or disease being treated. As used herein, the term “therapeutically effective amount” refers to an amount of a therapeutic agent which provides a desired therapeutic effect when administered to the target subject.

### Vectors

In certain embodiments, the therapeutic agent comprises an siRNA material. In some embodiments, the siRNA molecules described herein can be inserted into, or encoded by, vectors such as plasmids, viral vectors or other nucleic acid expression vectors for delivery to a cell. Preferably, the siRNA molecules are inserted into, or encoded by, viral vectors.

Viral vectors may be Herpesvirus (HSV) vectors, retroviral vectors, adenoviral vectors, adeno-associated viral (AAV) vectors, lentiviral vectors, and the like. In some specific embodiments, the viral vectors are AAV vector (e.g. AAV particles).

DNA expression plasmids can be used to stably express the siRNA duplexes or dsRNA of the present disclosure in cells and achieve long-term inhibition of target gene.

In some embodiments, the siRNA duplex targeting SOD1 gene may be encoded by a retroviral vector (See, e.g., U.S. Pat. Nos. 5,399,346; 5,124,263; 4,650,764 and 4,980,289; the content of each of which is incorporated herein by reference in their entirety).

Adenoviruses are eukaryotic DNA viruses that can be modified to efficiently deliver a nucleic acid to a variety of cell types in vivo, and have been used extensively in gene therapy protocols, including for targeting genes to neural cells. Various replication defective adenovirus and minimum adenovirus vectors have been described for nucleic acid therapeutics (See, e.g., PCT Patent Publication Nos. WO199426914, WO 199502697. WO199428152, WO199412649, WO199502697 and WO199622378; the content of each of which is incorporated by reference in their entirety). Such adenoviral vectors may also be used to deliver siRNA molecules of the present disclosure to cells.

### Adeno-Associated Viral (AAV) Vectors

An AAV is a dependent parvovirus. Like other parvoviruses, AAV is a single stranded, non-enveloped DNA virus, having a genome of about 5000 nucleotides in length containing two open reading frames that encode the proteins responsible for replication (Rep) and the structural protein of the capsid (Cap). The open reading frames are flanked by two Inverted Terminal Repeat (ITR) sequences, which serve as the origin of replication of viral genome. Furthermore, the AAV genome contains a packaging sequence, allowing packaging of the viral genome into an AAV capsid. The AAV vector requires co-helper (e.g., adenovirus) to undergo a productive infection in infected cells. In the absence of such helper functions, the AAV virions essentially enter host cells and integrate into cells' genome.

AAV vectors have been investigated for siRNA delivery because of its several unique features. These features include (i) ability to infect both dividing and non-dividing cells; (ii) a broad host range for infectivity, including human cells; (iii) wild-type AAV has never been associated with any disease and cannot replicate in infected cells; (iv) lack of cell-mediated immune response against the vector and (v) ability to integrate into a host chromosome or persist episomally, thereby creating potential for long-term expression. Moreover, infection with AAV vectors has minimal influence on changing the pattern of cellular gene expression (Stilwell and Samulski et al., Biotechniques, 2003, 34, 148).

Typically, AAV vectors for siRNA delivery may be recombinant viral vectors which are replication defective because of lacking sequences encoding functional Rep and Cap proteins in viral genome. In some cases, the defective AAV vectors may lack most of all coding sequences and essentially only contains one or two AAV ITR sequences and a packaging sequence.

AAV vectors may also comprise self-complementary AAV vectors (scAAVs), scAAV vectors contain both DNA strands which anneal together to form double stranded DNA. By skipping second strand synthesis, scAAVs allow for rapid expression in the cell.

Methods for producing/modifying AAV vectors are disclosed in the art such as pseudotyped AAV vectors (PCT Patent Publication Nos. WO200028004; WO200123001; WO2004112727; WO 2005005610 and WO 2005072364, the content of each of which is incorporated herein by reference in their entirety).

AAV vectors for delivering siRNA molecules into mammalian cells, may be prepared or derived from various serotypes of AAVs, including, but not limited to, AAV1, AAV2, AAV3, AAV4, AAV5, AAV6, AAV7, AAV8, AAV9, AAV9.47, AAV9(hu14), AAV10, AAV11, AAV12, AAVrh8, AAVrh10, AAV-DJ8 and AAV-DJ. In some cases, different serotypes of AAVs may be mixed together or with other types of viruses to produce chimeric AAV vectors.

In one embodiment, the AAV serotype is AAVrh10. In one embodiment, the AAV serotype is AAV9. In one embodiment, the AAV serotype is AAV2.

AAV vectors for siRNA delivery may be modified to enhance the efficiency of delivery. Such modified AAV vectors containing the siRNA expression cassette can be packaged efficiently and can be used to infect successfully the target cells at high frequency and with minimal toxicity.

In some embodiments, the AAV vector for delivering siRNA duplexes of the present disclosure may be a human serotype AAV vector. Such human AAV vector may be derived from any known serotype, e.g., from any one of serotypes AAV1-AAV11. As non-limiting examples, AAV vectors may be vectors comprising an AAV1-derived genome in an AAV1-derived capsid; vectors comprising an AAV2-derived genome in an AAV2-derived genome; vectors comprising an AAV4-derived genome in an AAV4 derived capsid; vectors comprising an AAV6-derived genome in an AAV6 derived capsid or vectors comprising an AAV9-derived genome in an AAV9 derived capsid.

In other embodiments, the AAV vector for delivering siRNA duplexes of the present disclosure may be a pseudotyped AAV vector which contains sequences and/or components originating from at least two different AAV serotypes. Pseudotyped AAV vectors may be vectors comprising an AAV genome derived from one AAV serotype and a Capsid protein derived at least in part from a different AAV serotype. As non-limiting examples, such pseudotyped AAV vectors may be vectors comprising an AAV2-derived genome in an AAV I-derived capsid; or vectors comprising an AAV2-derived genome in an AAV6-derived capsid; or vectors comprising an AAV2-derived genome in an AAV4-derived capsid; or an AAV2-derived genome in an AAV9-derived capsid. In like fashion, the present disclosure contemplates any hybrid or chimeric AAV particle.

In other embodiments, AAV vectors may be used for delivering siRNA molecules to the central nervous system (e.g., U.S. Pat. No. 6,180,613; the content of which is herein incorporated by reference in its entirety).

In some aspects, the AAV vector for delivering siRNA duplexes of the present disclosure may further comprise a modified capsid including peptides from non-viral origin. In other aspects, the AAV vector may contain a CNS specific chimeric capsid to facilitate the delivery of siRNA duplexes into the brain and the spinal cord. For example, an alignment of cap nucleotide sequences from AAV variants exhibiting CNS tropism may be constructed to identify variable region (VR) sequence and structure.

### AAV Particles Comprising Modulatory Polynucleotides

In one embodiment, the AAV particle comprises a viral genome with a payload region comprising a modulatory polynucleotide sequence. In such an embodiment, a viral genome encoding more than one polypeptide may be replicated and packaged into a viral particle. A target cell transduced with a viral particle comprising a modulatory polynucleotide may express the encoded sense and/or antisense sequences in a single cell.

In some embodiments, the AAV particles are useful in the field of medicine for the treatment, prophylaxis, palliation or amelioration of neurological diseases and/or disorders.

In one embodiment, the AAV particles comprising modulatory polynucleotide sequence which comprises a nucleic acid sequence encoding at least one siRNA molecule may be introduced into mammalian cells.

Where the AAV particle payload region comprises a modulatory polynucleotide, the modulatory polynucleotide may comprise sense and/or antisense sequences to knock down a target gene. The AAV viral genomes encoding modulatory polynucleotides described herein may be useful in the fields of human disease, viruses, infections veterinary applications and a variety of in vivo and in vitro settings.

In one embodiment, the AAV particle viral genome may comprise at least one inverted terminal repeat (ITR) region. In one embodiment, the AAV particle viral genome may comprises two inverted terminal repeat (ITR) regions. The ITR region(s) may, independently, have a length such as, but not limited to, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, and 175 nucleotides. The ITR region(s) may, independently, have a length such as, but not limited to 75-80, 75-85, 75-100, 80-85, 80-90, 80-105, 85-90, 85-95, 85-110, 90-95, 90-100, 90-115, 95-100, 95-105, 95-120, 100-105, 100-110, 100-125, 105-110, 105-115, 105-130, 110-115, 110-120, 110-135, 115-120, 115-125, 115-140, 120-125, 120-130, 120-145, 125-130, 125-135, 125-150, 130-135, 130-140, 130-155, 135-140, 135-145, 135-160, 140-145, 140-150, 140-165, 145-150, 145-155, 145-170, 150-155, 150-160, 150-175, 155-160, 155-165, 160-165, 160-170, 165-170, 165-175, and 170-175 nucleotides. As a non-limiting example, the viral genome comprises an ITR that is about 105 nucleotides in length. As a non-limiting example, the viral genome comprises an ITR that is about 141 nucleotides in length. As a non-limiting example, the viral genome comprises an ITR that is about 130 nucleotides in length.

In one embodiment, the AAV particle viral genome may comprise at least one multiple filler sequence region. The filler region(s) may, independently, have a length such as, but not limited to 50-100, 100-150, 150-200, 200-250, 250-300, 300-350, 350-400, 400-450, 450-500, 500-550, 550-600, 600-650, 650-700, 700-750, 750-800, 800-850, 850-900, 900-950, 950-1000, 1000-1050, 1050-1100, 1100-1150, 1150-1200, 1200-1250, 1250-1300, 1300-1350, 1350-1400, 1400-1450, 1450-1500, 1500-1550, 1550-1600, 1600-1650, 1650-1700, 1700-1750, 1750-1800, 1800-1850, 1850-1900, 1900-1950, 1950-2000, 2000-2050, 2050-2100, 2100-2150, 2150-2200, 2200-2250, 2250-2300, 2300-2350, 2350-2400, 2400-2450, 2450-2500, 2500-2550, 2550-2600, 2600-2650, 2650-2700, 2700-2750, 2750-2800, 2800-2850, 2850-2900, 2900-2950, 2950-3000, 3000-3050, 3050-3100, 3100-3150, 3150-3200, and 3200-3250 nucleotides. As non-limiting examples, the viral genome can comprise a filler region that is about 55 nucleotides in length, about 56 nucleotides in length, about 97 nucleotides in length, about 103 nucleotides in length, about 105 nucleotides in length, about 357 nucleotides in length, about 363 nucleotides in length, about 712 nucleotides in length, about 714 nucleotides in length, about 1203 nucleotides in length, about 1209 nucleotides in length, about 1512 nucleotides in length, about 1519 nucleotides in length, about 2395 nucleotides in length, about 2403 nucleotides in length, about 2405 nucleotides in length, about 3013 nucleotides in length or about 3021 nucleotides in length.

In one embodiment, the AAV particle viral genome may comprise at least one enhancer sequence region. The enhancer sequence region(s) may, independently, have a length such as, but not limited to 300-310, 300-325, 305-315, 310-320, 315-325, 320-330, 325-335, 325-350, 330-340, 335-345, 340-350, 345-355, 350-360, 350-375, 355-365, 360-370, 365-375, 370-380, 375-385, 375-400, 380-390, 385-395, and 390-400 nucleotides. As a non-limiting example, the viral genome comprises an enhancer region that is about 303 nucleotides in length. As a non-limiting example, the viral genome comprises an enhancer region that is about 382 nucleotides in length.

In one embodiment, the AAV particle viral genome may comprise at least one promoter sequence region. The promoter sequence region(s) may, independently, have a length such as, but not limited to 4-10, 10-20, 10-50, 20-30, 30-40, 40-50, 50-60, 50-100, 60-70, 70-80, 80-90, 90-100, 100-110, 100-150, 110-120, 120-130, 130-140, 140-150, 150-160, 150-200, 160-170, 170-180, 180-190, 190-200, 200-210, 200-250, 210-220, 220-230, 230-240, 240-250, 250-260, 250-300, 260-270, 270-280, 280-290, 290-300, 300-310, 300-350, 310-320, 320-330, 330-340, 340-350, 350-360, 350-400, 360-370, 370-380, 380-390, 390-400, 400-410, 400-450, 410-420, 420-430, 430-440, 440-450, 450-460, 450-500, 460-470, 470-480, 480-490, 490-500, 500-510, 500-550, 510-520, 520-530, 530-540, 540-550, 550-560, 550-600, 560-570, 570-580, 580-590, and 590-600 nucleotides. As non-limiting examples, the viral genome can comprise a promoter region that is about 4 nucleotides in length, about 17 nucleotides in length, about 204 nucleotides in length, about 219 nucleotides in length, about 260 nucleotides in length, about 303 nucleotides in length, about 382 nucleotides in length or about 588 nucleotides in length.

In one embodiment, the AAV particle viral genome may comprise at least one exon sequence region. The exon region(s) may, independently, have a length such as, but not limited to 2-10, 5-10, 5-15, 10-20, 10-30, 10-40, 15-20, 15-25, 20-30, 20-40, 20-50, 25-30, 25-35, 30-40, 30-50, 30-60, 35-40, 35-45, 40-50, 40-60, 40-70, 45-50, 45-55, 50-60, 50-70, 50-80, 55-60, 55-65, 60-70, 60-80, 60-90, 65-70, 65-75, 70-80, 70-90, 70-100, 75-80, 75-85, 80-90, 80-100, 80-110, 85-90, 85-95, 90-100, 90-110, 90-120, 95-100, 95-105, 100-110, 100-120, 100-130, 105-110, 105-115, 110-120, 110-130, 110-140, 115-120, 115-125, 120-130, 120-140, 120-150, 125-130, 125-135, 130-140, 130-150, 135-140, 135-145, 140-150, and 145-150 nucleotides. As a non-limiting example, the viral genome comprises an exon region that is about 53 nucleotides in length. As a non-limiting example, the viral genome comprises an exon region that is about 134 nucleotides in length.

In one embodiment, the AAV particle viral genome may comprise at least one intron sequence region. The intron region(s) may, independently, have a length such as, but not limited to 25-35, 25-50, 35-45, 45-55, 50-75, 55-65, 65-75, 75-85, 75-100, 85-95, 95-105, 100-125, 105-115, 115-125, 125-135, 125-150, 135-145, 145-155, 150-175, 155-165, 165-175, 175-185, 175-200, 185-195, 195-205, 200-225, 205-215, 215-225, 225-235, 225-250, 235-245, 245-255, 250-275, 255-265, 265-275, 275-285, 275-300, 285-295, 295-305, 300-325, 305-315, 315-325, 325-335, 325-350, and 335-345 nucleotides. As a non-limiting example, the viral genome comprises an intron region that is about 32 nucleotides in length. As a non-limiting example, the viral genome comprises an intron region that is about 172 nucleotides in length. As a non-limiting example, the viral genome comprises an intron region that is about 201 nucleotides in length. As a non-limiting example, the viral genome comprises an intron region that is about 347 nucleotides in length.

In one embodiment, the AAV particle viral genome may comprise at least one polyadenylation signal sequence region. The polyadenylation signal region sequence region(s) may, independently, have a length such as, but not limited to 4-10, 10-20, 10-50, 20-30, 30-40, 40-50, 50-60, 50-100, 60-70, 70-80, 80-90, 90-100, 100-110, 100-150, 110-120, 120-130, 130-140, 140-150, 150-160, 150-200, 160-170, 170-180, 180-190, 190-200, 200-210, 200-250, 210-220, 220-230, 230-240, 240-250, 250-260, 250-300, 260-270, 270-280, 280-290, 290-300, 300-310, 300-350, 310-320, 320-330, 330-340, 340-350, 350-360, 350-400, 360-370, 370-380, 380-390, 390-400, 400-410, 400-450, 410-420, 420-430, 430-440, 440-450, 450-460, 450-500, 460-470, 470-480, 480-490, 490-500, 500-510, 500-550, 510-520, 520-530, 530-540, 540-550, 550-560, 550-600, 560-570, 570-580, 580-590, and 590-600 nucleotides. As a non-limiting example, the viral genome comprises a polyadenylation signal sequence region that is about 127 nucleotides in length. As a non-limiting example, the viral genome comprises a polyadenylation signal sequence region that is about 225 nucleotides in length. As a non-limiting example, the viral genome comprises a polyadenylation signal sequence region that is about 476 nucleotides in length. As a non-limiting example, the viral genome comprises a polyadenylation signal sequence region that is about 477 nucleotides in length.

In one embodiment, the AAV particle viral genome comprises more than one polyA signal sequence region.

Non-limiting examples of ITR to ITR sequences of AAV particles comprising a viral genome with a payload region comprising a modulatory polynucleotide sequence are described in Table 1A.

TABLE 1A
ITR to ITR Sequences of AAV Particles, H1.mir.104-
788.2 (with lentivirus derived filler) comprising
Modulatory Polynucleotides

| ITR to ITR | ITR to ITR | Modulatory Polynucleotide |
|---|---|---|
| Construct Name | SEQ ID NO | SEQ ID NO |
| H1.mir.104-788.2 with | 2 | 1 |
| lentivirus derived filler | | |
| (VOYSOD16) | | |

Non-limiting examples of ITR to ITR sequences of AAV particles comprising a viral genome with a payload region comprising a modulatory polynucleotide sequence are described in Table 1B. Table 1B provides ITR to ITR sequence of H1.mir104-788.2 with albumin derived filler. Also provided in Table 1B are the components that comprise the ITR to ITR sequence.

TABLE 1B
ITR to ITR of AAV Particles, H1.mir104-788.2
(with albumin derived filler) comprising Modulatory
Polynucleotides and its components

| Description | SEQ ID NO. |
|---|---|
| ITR to ITR of H1.mir104-788.2 | 3 |
| with albumin derived filler | |

ITR-ITR Components of H1.mir104-788.2 (with albumin
derived filler)

| | |
|---|---|
| 5′ ITR | 4 |
| Albumin derived filler | 5 |
| H1 promoter | 6 |
| Modulatory Polynucleotide | 1 |
| (SOD1-miR104-788.2) | |
| rBGpA | 7 |
| 3′ ITR | 8 |

In one embodiment, the AAV particle comprises a viral genome which comprises a sequence which has a percent identity to SEQ ID NO: 2. The viral genome may have 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99% or 100% identity to SEQ ID NO: 2. The viral genome may have 1-10%, 10-20%, 30-40%, 50-60%, 50-70%, 50-80%, 50-90%, 50-99%, 50-100%, 60-70%, 60-80%, 60-90%, 60-99%, 60-100%, 70-80%, 70-90%, 70-99%, 70-100%, 80-85%, 80-90%, 80-95%, 80-99%, 80-100%, 90-95%, 90-99%, or 90-100% to SEQ ID NO: 2. As a non-limiting example, the viral genome comprises a sequence which as 80% identity to SEQ ID NO: 2. As another non-limiting example, the viral genome comprises a sequence which as 85% identity to SEQ ID NO: 2. As another non-limiting example, the viral genome comprises a sequence which as 90% identity to SEQ ID NO: 2. As another non-limiting example, the viral genome comprises a sequence which as 95% identity to SEQ ID NO: 2. As another non-limiting example, the viral genome comprises a sequence which as 99% identity to SEQ ID NO: 2.

In one embodiment, the AAV particle comprises a viral genome which comprises a sequence which has a percent identity to SEQ ID NO: 3. The viral genome may have 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99% or 100% identity to SEQ ID NO: 3. The viral genome may have 1-10%, 10-20%, 30-40%, 50-60%, 50-70%, 50-80%, 50-90%, 50-99%, 50-100%, 60-70%, 60-80%, 60-90%, 60-99%, 60-100%, 70-80%, 70-90%, 70-99%, 70-100%, 80-85%, 80-90%, 80-95%, 80-99%, 80-100%, 90-95%, 90-99%, or 90-100% to SEQ ID NO: 3. As a non-limiting example, the viral genome comprises a sequence which as 80% identity to SEQ ID NO: 3. As another non-limiting example, the viral genome comprises a sequence which as 85% identity to SEQ ID NO: 3. As another non-limiting example, the viral genome comprises a sequence which as 90% identity to SEQ ID NO: 3. As another non-limiting example, the viral genome comprises a sequence which as 95% identity to SEQ ID NO: 3. As another non-limiting example, the viral genome comprises a sequence which as 99% identity to SEQ ID NO: 3.

In one aspect, the sense and antisense strands of a siRNA duplex encoded by a SOD1 targeting polynucleotide are typically linked by a short spacer sequence leading to the expression of a stem-loop structure termed short hairpin RNA (shRNA). The hairpin is recognized and cleaved by Dicer, thus generating mature siRNA molecules.

According to the present disclosure, AAV vectors comprising the nucleic acids of the siRNA molecules targeting SOD1 mRNA are produced, the AAV vectors may be AAV1, AAV2, AAV3, AAV4, AAV5, AAV6, AAV7, AAV8. AAV9, AAV9.47, AAV9(hu14), AAV10. AAV11, AAV12, AAVrh8, AAVrh10, AAV-DJ8 and AAV-DJ, and variants thereof.

In some embodiments, the siRNA duplexes or dsRNA of the present disclosure when expressed suppress (or degrade) target mRNA (i.e. SOD1). Accordingly, the siRNA duplexes or dsRNA encoded by a SOD1 targeting polynucleotide can be used to substantially inhibit SOD1 gene expression in a cell, for example a motor neuron. In some aspects, the inhibition of SOD1 gene expression refers to an inhibition by at least about 20%, preferably by at least about 30%, 40%, 50%, 60%, 70%, 80%, 85%, 90%, 95% and 100%. Accordingly, the protein product of the targeted gene may be inhibited by at least about 20%, preferably by at least about 30%, 40%, 50%, 60%, 70%, 80%, 85%, 90%, 95% and 100%. The SOD1 gene can be either a wild type gene or a mutated SOD1 gene with at least one mutation. Accordingly, the protein is either wild type protein or a mutated polypeptide with at least one mutation.

### Pharmaceutical Compositions and Formulation

In addition to the pharmaceutical compositions, e.g., siRNA duplexes (including the encoding plasmids or expression vectors, such as viruses, e.g., AAV) to be delivered, provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

In some embodiments, compositions are administered to humans, human patients or subjects. For the purposes of the present disclosure, the phrase “active ingredient” generally refers either to synthetic siRNA duplexes or to the viral vector carrying the siRNA duplexes, or to the siRNA molecule delivered by a viral vector as described herein.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered.

The siRNA duplexes or viral vectors encoding them can be formulated using one or more excipients to: (1) increase stability; (2) increase cell transfection or transduction; (3) permit the sustained or delayed release; or (4) alter the biodistribution (e.g., target the viral vector to specific tissues or cell types such as brain and motor neurons).

Formulations of the present disclosure can include, without limitation, saline, lipidoids, liposomes, lipid nanoparticles, polymers, lipoplexes, core-shell nanoparticles, peptides, proteins, cells transfected with viral vectors (e.g., for transplantation into a subject), nanoparticle mimics and combinations thereof. Further, the viral vectors of the present disclosure may be formulated using self-assembled nucleic acid nanoparticles.

Formulations of the pharmaceutical compositions described herein may be prepared by any method known or hereafter developed in the art of pharmacology. In general, such preparatory methods include the step of associating the active ingredient with an excipient and/or one or more other accessory ingredients.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In some embodiments, the formulations described herein may contain at least one SOD1 targeting polynucleotide. As a non-limiting example, the formulations may contain 1, 2, 3, 4 or 5 polynucleotide that target SOD1 gene at different sites.

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia. and/or the International Pharmacopoeia.

Excipients, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21^{st} Edition. A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

In some embodiments, the formulations may comprise at least one inactive ingredient. As used herein, the term “inactive ingredient” refers to one or more inactive agents included in formulations. In some embodiments, all, none or some of the inactive ingredients which may be used in the formulations of the present disclosure may be approved by the US Food and Drug Administration (FDA).

Formulations of viral vectors carrying SOD1 targeting polynucleotides disclosed herein may include cations or anions. In one embodiment, the formulations include metal cations such as, but not limited to, Zn2+, Ca2+, Cu2+, Mg+ and combinations thereof.

As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977); the content of each of which is incorporated herein by reference in their entirety.

The term “pharmaceutically acceptable solvate,” as used herein, means a compound of the disclosure wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

According to the present disclosure, the SOD1 targeting polynucleotides, or AAV vectors comprising the same, may be formulated for CNS delivery. Agents that cross the brain blood barrier may be used. For example, some cell penetrating peptides that can target siRNA molecules to the brain blood barrier endothelium may be used to formulate the siRNA duplexes targeting SOD1 gene (e.g., Mathupala, Expert Opin Ther Pat., 2009, 19, 137-140; the content of which is incorporated herein by reference in its entirety).

In one embodiment, the AAV particles of the disclosure may be formulated in PBS, in combination with an ethylene oxide/propylene oxide copolymer (also known as pluronic or poloxamer).

In one embodiment, the AAV particles of the disclosure may be formulated in PBS with 0.001% pluronic acid (F-68) (poloxamer 188) at a pH of about 7.0.

In one embodiment, the AAV particles of the disclosure may be formulated in PBS with 0.001% pluronic acid (F-68) (poloxamer 188) at a pH of about 7.3.

In one embodiment, the AAV particles of the disclosure may be formulated in PBS with 0.001% pluronic acid (F-68) (poloxamer 188) at a pH of about 7.4.

In one embodiment, the AAV particles of the disclosure may be formulated in a solution comprising sodium chloride, sodium phosphate and an ethylene oxide/propylene oxide copolymer.

In one embodiment, the AAV particles of the disclosure may be formulated in a solution comprising sodium chloride, sodium phosphate dibasic, sodium phosphate monobasic and poloxamer 188/pluronic acid (F-68).

### IV. Methods of Treatment

Provided in the present disclosure are methods for introducing the SOD1 targeting polynucleotides described herein into cells, the method comprising introducing into said cells any of the polynucleotides in an amount sufficient for degradation of target SOD1 mRNA to occur. In some aspects, the cells may be stem cells, neurons such as motor neurons, muscle cells and glial cells such as astrocytes.

Described here are methods for delivering AAV particles to the spinal cord, for the treatment of disorders associated with the spinal cord, such as, but not limited to motor neuron disease (e.g., ALS). In one embodiment, these methods result in trans-synaptic transmission.

Disclosed in the present disclosure are also methods for treating ALS associated with abnormal SOD1 function in a subject in need of treatment. The method optionally comprises administering to the subject a therapeutically effective amount of a composition comprising or encoding at least one siRNA duplex targeting SOD1 gene. Said siRNA duplex will silence SOD1 gene expression and inhibit SOD1 protein production and reduce one or more symptoms of ALS in the subject such that ALS is therapeutically treated.

In some embodiments, the SOD1 targeting polynucleotide of the present disclosure is administered to the central nervous system of the subject. In other embodiments, the siRNA duplex of the present disclosure or the composition comprising it is administered to the muscles of the subject

In particular, the SOD1 targeting polynucleotides may be delivered into specific types of targeted cells, including motor neurons; glial cells including oligodendrocyte, astrocyte and microglia, and/or other cells surrounding neurons such as T cells. Studies in human ALS patients and animal SOD1 ALS model implicated that glial cells play an early role in the dysfunction and death of ALS neurons. Normal SOD1 in the surrounding, protective glial cells can prevent the motor neurons from dying even though mutant SOD1 is present in motor neurons (e.g., reviewed by Philips and Rothstein, Exp. Neurol., 2014, May 22. pii: S0014-4886(14)00157-5; the content of which is incorporated herein by reference in its entirety).

In some specific embodiments, at least one siRNA duplex targeting SOD1 gene used as a therapy for ALS is inserted in a viral vector, such as an AAV vector.

In some embodiments, the present composition is administered as a single therapeutic or combination therapeutics for the treatment of ALS.

The viral vectors comprising or encoding siRNA duplexes targeting SOD1 gene may be used in combination with one or more other therapeutic, agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent.

Therapeutic agents that may be used in combination with the SOD1 targeting polynucleotides of the present disclosure can be small molecule compounds which are antioxidants, anti-inflammatory agents, anti-apoptosis agents, calcium regulators, antiglutamatergic agents, structural protein inhibitors, and compounds involved in metal ion regulation.

Compounds used in combination for treating ALS may include, but are not limited to, agents that reduce oxidative stress, such as free-radical scavengers, or Radicava (edaravone), antiglutamatergic agents: Riluzole, Topiramate, Talampanel, Lamotrigine, Dextromethorphan, Gabapentin and AMPA antagonist; Anti-apoptosis agents: Minocycline, Sodium phenylbutyrate and Arimoclomol; Anti-inflammatory agent: ganglioside, Celecoxib, Cyclosporine, Azathioprine, Cyclophosphamide, Plasmaphoresis, Glatiramer acetate and thalidomide; Ceftriaxone (Berry et al., Plos One, 2013, 8(4)): Beat-lactam antibiotics; Pramipexole (a dopamine agonist) (Wang et al., Amyotrophic Lateral Scler., 2008, 9(1), 50-58); Nimesulide in U.S. Patent Publication No. 20060074991: Diazoxide disclosed in U.S. Patent Publication No. 20130143873): pyrazolone derivatives disclosed in US Patent Publication No. 20080161378: free radical scavengers that inhibit oxidative stress-induced cell death, such as bromocriptine (US. Patent Publication No. 20110105517); phenyl carbamate compounds discussed in PCT Patent Publication No. 2013100571; neuroprotective compounds disclosed in U.S. Pat. Nos. 6,933,310 and 8,399,514 and US Patent Publication Nos. 20110237907 and 20140038927; and glycopeptides taught in U.S. Patent Publication No. 20070185012; the content of each of which is incorporated herein by reference in their entirety.

Therapeutic agents that may be used in combination therapy with the siRNA duplexes targeting SOD1 gene of the present disclosure may be hormones or variants that can protect neuron loss, such as adrenocorticotropic hormone (ACTH) or fragments thereof (e.g., U.S. Patent Publication No. 20130259875); Estrogen (e.g., U.S. Pat. Nos. 6,334,998 and 6,592,845); the content of each of which is incorporated herein by reference in their entirety.

Neurotrophic factors may be used in combination therapy with the siRNA duplexes targeting SOD1 gene of the present disclosure for treating ALS. Generally, a neurotrophic factor is defined as a substance that promotes survival, growth, differentiation, proliferation and/or maturation of a neuron, or stimulates increased activity of a neuron. In some embodiments, the present methods further comprise delivery of one or more trophic factors into the subject in need of treatment. Trophic factors may include, but are not limited to, IGF-I, GDNF, BDNF, CTNF, VEGF, Colivelin, Xaliproden, Thyrotrophin-releasing hormone and ADNF, and variants thereof.

In one aspect, the AAV vector comprising at least one siRNA duplex targeting SOD1 gene may be co-administered with AAV vectors expressing neurotrophic factors such as AAV-IGF-I (Vincent et al., Neuromolecular medicine, 2004, 6, 79-85; the content of which is incorporated herein by reference in its entirety) and AAV-GDNF (Wang et al., J Neurosci., 2002, 22, 6920-6928; the content of which is incorporated herein by reference in its entirety).

In some embodiments, the composition of the present disclosure for treating ALS is administered to the subject in need intravenously, intramuscularly, subcutaneously, intraperitoneally, intrathecally, intraparenchymally (CNS, brain, and/or spinal cord) and/or intraventricularly, allowing the siRNA duplexes or vectors comprising the siRNA duplexes to pass through one or both the blood-brain barrier and the blood spinal cord barrier. In some aspects, the method includes administering (e.g., intraparenchymally administering, intraventricularly administering and/or intrathecally administering) directly to the central nervous system (CNS) of a subject (using, e.g., an infusion pump and/or a delivery scaffold) a therapeutically effective amount of a composition comprising at least one siRNA duplex targeting SOD1 gene or AAV vectors comprising at least one siRNA duplex targeting SOD1 gene, silencing/suppressing SOD1 gene expression, and reducing one or more symptoms of ALS in the subject such that ALS is therapeutically treated.

In some embodiments, the composition of the present disclosure for treating ALS is administered to the subject in need intraparenchymally (CNS, brain, and/or spinal cord), allowing the siRNA duplexes or vectors comprising the siRNA duplexes to pass through one or both the blood-brain barrier and the blood spinal cord barrier.

In certain aspects, the symptoms of ALS including motor neuron degeneration, muscle weakness, muscle atrophy, the stiffness of muscle, difficulty in breathing, slurred speech, fasciculation development, frontotemporal dementia and/or premature death are improved in the subject treated. In other aspects, the composition of the present disclosure is applied to one or both of the brain and the spinal cord. In other aspects, one or both of muscle coordination and muscle function are improved. In other aspects, the survival of the subject is prolonged.

### Administration

A therapeutic agent of the present disclosure (e.g. SOD1 targeting polynucleotide) of the present disclosure may be administered by any route which results in a therapeutically effective outcome. These include, but are not limited to intraparenchymal (into brain tissue), intraparenchymal (spinal cord), intraparenchymal (CNS), enteral (into the intestine), gastroenteral, epidural (into the dura matter), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavemous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracistemal (within the cistema magna cerebellomedularis), intracorneal (within the cornea), dental intracornal, intracoronary (within the coronary arteries), intracorporus cavemosum (within the dilatable spaces of the corporus cavemosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subeonjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis, intrastriatal (within the striatum) infusionor spinal.

In specific embodiments, compositions including AAV vectors comprising at least one SOD1 targeting polynucleotide may be administered in a way which allows them to enter the central nervous system and penetrate into motor neurons.

In some embodiments, the therapeutics of the present disclosure may be administered by muscular injection. Rizvanov et al. demonstrated for the first time that siRNA molecules, targeting mutant human SOD1 mRNA, is taken up by the sciatic nerve, retrogradely transported to the perikarya of motor neurons, and inhibits mutant SOD1 mRNA in SOD1^{G93A} transgenic ALS mice (Rizvanov A A et al., Exp. Brain Res., 2009, 195(1), 1-4; the content of which is incorporated herein by reference in its entirety). Another study also demonstrated that muscle delivery of AAV expressing small hairpin RNAs (shRNAs) against the mutant SOD1 gene, led to significant mutant SOD1 knockdown in the muscle as well as innervating motor neurons (Towne C et al., Mol Ther., 2011; 19(2): 274-283; the content of which is incorporated herein by reference in its entirety).

In some embodiments, AAV vectors that express siRNA duplexes of the present disclosure may be administered to a subject by peripheral injections and/or intranasal delivery. It was disclosed in the art that the peripheral administration of AAV vectors for siRNA duplexes can be transported to the central nervous system, for example, to the motor neurons (e.g., U.S. Patent Publication Nos. 20100240739; and 20100130594; the content of each of which is incorporated herein by reference in their entirety).

In other embodiments, compositions comprising at least one siRNA duplex of the disclosure may be administered to a subject by intracranial delivery (See, e.g., U.S. Pat. No. 8,119,611; the content of which is incorporated herein by reference in its entirety).

The SOD1 targeting polynucleotides of the present disclosure may be administered in any suitable forms, either as a liquid solution or suspension, as a solid form suitable for liquid solution or suspension in a liquid solution. They may be formulated with any appropriate and pharmaceutically acceptable excipient.

The SOD1 targeting polynucleotides of the present disclosure may be administered in a “therapeutically effective” amount, i.e., an amount that is sufficient to alleviate and/or prevent at least one symptom associated with the disease, or provide improvement in the condition of the subject.

In some embodiments, the pharmaceutical compositions of the present disclosure may be administered by intraparenchymal injection or infusion. As used herein, “injection” and “infusion” may be used interchangeably and indicate the same. As a non-limiting example, the pharmaceutical compositions of the present disclosure may be administered to a subject by intraparenchymal injection. In one embodiment, the intraparenchymal injection may be a spinal intraparenchymal injection, wherein the pharmaceutical compositions may be administered directly to the tissue of the spinal cord. In one embodiment, the intraparenchymal injection may be a CNS (central nervous system) intraparenchymal injection wherein the pharmaceutical compositions may be administered directly to the tissue of the CNS.

In one embodiment, the pharmaceutical compositions of the present disclosure may be administered to the cistema magna in a therapeutically effective amount to transduce spinal cord motor neurons and/or astrocytes.

In one embodiment, the pharmaceutical compositions of the present disclosure may be administered by intrastriatal infusion.

In some embodiments, the pharmaceutical compositions of the present disclosure may be administered by intraparenchymal injection as well as by another route of administration described herein.

In some embodiments, the pharmaceutical compositions of the present disclosure may be administered by intraparenchymal injection and intrathecal injection. In one embodiment, the pharmaceutical compositions of the present disclosure may be administered by intraparenchymal injection and intrastriatal injection.

In one embodiment, the AAV particle described herein is administered via intraparenchymal (IPa) infusion at any level of the spinal cord, at a single or at multiple sites, at a volume of more than 1 uL. In one embodiment, a volume of 1 uL-100 uL is administered. In one embodiment, a volume of 1 uL-240 uL is administered. In one embodiment, a volume of 1 uL-240 uL is administered. In one embodiment, a volume of 1 uL-220 uL is administered. In one embodiment, a volume of between 1 uL-200 uL is administered. In one embodiment, a volume of 1 uL-180 uL is administered. In one embodiment, a volume of 1 uL-160 uL is administered. In one embodiment, a volume of 1 uL-150 uL is administered. In one embodiment, a volume of 1 uL-140 uL is administered. In one embodiment, a volume of 1 uL-130 uL is administered. In one embodiment, a volume of 1 uL-120 uL is administered. In one embodiment, a volume of 1 uL-110 uL is administered. In one embodiment, a volume of 1 uL-90 uL is administered. In one embodiment, a volume of between 1 uL-80 uL is administered. In one embodiment, a volume of 1 uL-70 uL is administered. In one embodiment, a volume of 1 uL-60 uL is administered. In one embodiment, a volume of 1 uL-50 uL is administered. In one embodiment, a volume of 1 uL-40 uL is administered. In one embodiment, a volume of 1 uL-30 uL is administered. In one embodiment, a volume of 1 uL-20 uL is administered. In one embodiment, a volume of 10 uL-20 uL is administered. In one embodiment, a volume of 10 uL-30 uL is administered. In one embodiment, a volume of 10 uL-40 uL is administered. In one embodiment, a volume of 10 uL-50 uL is administered. In one embodiment, a volume of 10 uL-60 uL is administered. In one embodiment, a volume of 10 uL-80 uL is administered. In one embodiment, a volume of 10 uL-90 uL is administered. In one embodiment, a volume of 20 uL-240 uL is administered. In one embodiment, a volume of 20 uL-200 uL is administered. In one embodiment, a volume of 20 uL-180 uL is administered. In one embodiment, a volume of 20 uL-150 uL is administered. In one embodiment, a volume of 20 uL-120 uL is administered. In one embodiment, a volume of 20 uL-100 uL is administered. In one embodiment, a volume of 20 uL-80 uL is administered. In one embodiment, a volume of 20 uL-60 uL is administered. In one embodiment, a volume of 20 uL-50 uL is administered. In one embodiment, a volume of 20 uL-40 uL is administered. In one embodiment, a volume of 20 uL-30 uL is administered. In one embodiment, a volume of 50 uL-200 uL is administered. In one embodiment, a volume of 50 uL-180 uL is administered. In one embodiment, a volume of 50 uL-150 uL is administered. In one embodiment, a volume of 50 uL-100 uL is administered. In one embodiment, a volume of 50 uL-80 uL is administered. In one embodiment, a volume of 50 uL-70 uL is administered. In one embodiment, a volume of 100 uL-240 uL is administered. In one embodiment, a volume of 100 uL-200 uL is administered. In one embodiment, a volume of 100 uL-180 uL is administered. In one embodiment, a volume of 100 uL-150 uL is administered.

In one embodiment, the AAV particle described herein is administered via intraparenchymal (IPa) infusion at C1, C2, C3, C4, C5, C6, C7, and/or LI.

In one embodiment, the AAV particle described herein is administered via intraparenchymal (IPa) infusion at C1. In one embodiment, the AAV particle described herein is administered via intraparenchymal (IPa) infusion at C2. In one embodiment, the AAV particle described herein is administered via intraparenchymal (IPa) infusion at C3. In one embodiment, the AAV particle described herein is administered via intraparenchymal (IPa) infusion at C4. In one embodiment, the AAV particle described herein is administered via intraparenchymal (IPa) infusion at C5. In one embodiment, the AAV particle described herein is administered via intraparenchymal (IPa) infusion at C6. In one embodiment, the AAV particle described herein is administered via intraparenchymal (IPa) infusion at C7.

In one embodiment, the AAV particle described herein is administered via intraparenchymal (IPa) infusion at two sites. In one embodiment, the AAV particle described herein is administered via intraparenchymal (IPa) infusion at C1 and C2. In one embodiment, the AAV particle described herein is administered via intraparenchymal (IPa) infusion at C1 and C3. In one embodiment, the AAV particle described herein is administered via intraparenchymal (IPa) infusion at C1 and C4. In one embodiment, the AAV particle described herein is administered via intraparenchymal (IPa) infusion at C1 and C5. In one embodiment, the AAV particle described herein is administered via intraparenchymal (IPa) infusion at C1 and C6. In one embodiment, the AAV particle described herein is administered via intraparenchymal (IPa) infusion at C1 and C7.

In one embodiment, the AAV particle described herein is administered via intraparenchymal (IPa) infusion at two sites. In one embodiment, the AAV particle described herein is administered via intraparenchymal (IPa) infusion at C2 and C3. In one embodiment, the AAV particle described herein is administered via intraparenchymal (IPa) infusion at C2 and C4. In one embodiment, the AAV particle described herein is administered via intraparenchymal (IPa) infusion at C2 and C5. In one embodiment, the AAV particle described herein is administered via intraparenchymal (IPa) infusion at C2 and C6. In one embodiment, the AAV particle described herein is administered via intraparenchymal (IPa) infusion at C2 and C7.

In one embodiment, the AAV particle described herein is administered via intraparenchymal (IPa) infusion at two sites. In one embodiment, the AAV particle described herein is administered via intraparenchymal (IPa) infusion at C3 and C4. In one embodiment, the AAV particle described herein is administered via intraparenchymal (IPa) infusion at C3 and C5. In one embodiment, the AAV particle described herein is administered via intraparenchymal (IPa) infusion at C3 and C6. In one embodiment, the AAV particle described herein is administered via intraparenchymal (IPa) infusion at C3 and C7.

In one embodiment, the AAV particle described herein is administered via intraparenchymal (IPa) infusion at two sites. In one embodiment, the AAV particle described herein is administered via intraparenchymal (IPa) infusion at C4 and C5. In one embodiment, the AAV particle described herein is administered via intraparenchymal (IPa) infusion at C4 and C6. In one embodiment, the AAV particle described herein is administered via intraparenchymal (IPa) infusion at C4 and C7.

In one embodiment, the AAV particle described herein is administered via intraparenchymal (IPa) infusion at two sites. In one embodiment, the AAV particle described herein is administered via intraparenchymal (IPa) infusion at C5 and C6. In one embodiment, the AAV particle described herein is administered via intraparenchymal (IPa) infusion at C5 and C7.

In one embodiment, the AAV particle described herein is administered via intraparenchymal (IPa) infusion at two sites. In one embodiment, the AAV particle described herein is administered via spinal cord infusion at two sites. In another embodiment, the AAV particle described herein comprises administration at level C3 or C5 of the spinal cord. In yet another embodiment, the AAV particle described herein are administered at levels C3 and C5 of the spinal cord. In one embodiment, the AAV particle described herein is administered via intraparenchymal (IPa) infusion at C6 and C7 of the spinal cord.

The intraparenchymal (IPa) infusion may be for 1, 2, 3, 4, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more than 60 minutes. As a non-limiting example, the infusion is for 10 minutes. As a non-limiting example, the infusion is for 11 minutes. As a non-limiting example, the infusion is for 12 minutes. As a non-limiting example, the infusion is for 13 minutes. As a non-limiting example, the infusion is for 14 minutes. As a non-limiting example, the infusion is for 15 minutes.

The intraparenchymal (IPa), e.g., spinal cord, infusion may be, independently, a dose volume of 5, 10, 15, 20, 25, 30, 35, 40, 45, 50, 55, 60 or more than 60 uL. As a non-limiting example, the dose volume is 20 uL. As a non-limiting example, the dose volume is 25 uL. As a non-limiting example, the dose volume is 30 uL. As a non-limiting example, the dose volume is 35 uL. As a non-limiting example, the dose volume is 40 uL. As a non-limiting example, the dose volume is 45 uL. As a non-limiting example, the dose volume is 50 uL. As a non-limiting example, the dose volume is 60 uL.

In one embodiment, the dose volume is 5 uL-60 uL per site of administration. In another embodiment, the dose volume is 25 uL-40 uL per site of administration. In one embodiment, the dose volume is 5 uL-60 uL for administration to level C3, C5, C6, or C7 of the spinal cord. In one embodiment, the dose volume is 5 uL-60 uL for administration to level C3 of the spinal cord. In another embodiment, the dose volume is 5 uL-60 uL for administration to level C5 of the spinal cord. In yet another embodiment, the dose volume is 5 uL-60 uL for administration to level C3 of the spinal cord and the dose volume for administration to level C5 of the spinal cord is 5 uL-60 uL. In one embodiment, the dose volume is 25 uL-40 uL for administration to level C3, C5, C6, or C7 of the spinal cord. In one embodiment, the dose volume is 25 uL-40 uL for administration to level C3 of the spinal cord. In another embodiment, the dose volume is 25 uL-40 uL for administration to level C5 of the spinal cord. In yet another embodiment, the dose volume is 25 uL-40 uL for administration to level C3 of the spinal cord and the dose volume for administration to level C5 of the spinal cord is 25 uL-40 uL.

The intraparenchymal (IPa), e.g., spinal cord, infusion may be at an injection rate of 1, 2, 3, 4, 5, 6, 7, 8, 9, 10, 11, 12, 13, 14, 15, or more than 15 uL/min. As a non-limiting example, the injection rate is 5 uL/min.

The intraparenchymal (IPa), e.g., spinal cord, infusion may be at a dose between about 1×10⁶ VG and about 1×10¹⁶ VG. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 4.1×10¹¹, 4.2×10¹¹, 4.3×10¹¹, 4.4×10¹¹, 4.5×10¹¹, 4.6×10¹¹, 4.7×10¹¹, 4.8×10¹¹, 4.9×10¹¹, 5×10¹¹, 6×10¹¹, 6.1×10¹¹, 6.2×10¹¹, 6.3×10¹¹, 6.4×10¹¹, 6.5×10¹¹, 6.6×10¹¹, 6.7×10¹¹, 6.8×10¹¹, 6.9×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 3×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 8.1×10¹², 8.2×10¹², 8.3×10¹², 8.4×10¹², 8.5×10¹², 8.6×10¹², 8.7×10¹², 8.8×10¹², 8.9×10¹², 9×10¹², 1×10¹³, 2×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG. As a non-limiting example, the dose is 4×10¹¹ VG. As a non-limiting example, the dose is 5.0×10¹¹ VG. As a non-limiting example, the dose is 5.1×10¹¹ VG. As a non-limiting example, the dose is 6.6×10¹¹ VG. As a non-limiting example, the dose is 8.0×10¹¹ VG. As a non-limiting example, the dose is 8.1×10¹¹ VG. As a non-limiting example, the dose is 1.0×10¹² VG. As a non-limiting example, the dose is 1.1×10¹² VG. As a non-limiting example, the dose is 1.2×10¹² VG. As a non-limiting example, the dose is 1.3×10¹² VG. As a non-limiting example, the dose is 1.0×10¹⁰ vg-1.0×10¹² VG. As a non-limiting example, the dose is 5.0×10¹¹ vg-8.0×10¹¹ VG.

In one embodiment, the intraparenchymal (IPa), e.g., spinal cord, infusion may be between about 1.0×10¹³ VG/ml and about 3×10¹³ VG/ml. In another embodiment, the intraparenchymal (IPa), e.g., spinal cord, infusion is 1.5×10¹³ VG/ml-3.0×10¹³ VG/ml. In yet another embodiment, the intraparenchymal (IPa), e.g., spinal cord, infusion is 1.8×10¹³ VG/ml-2.5×10¹³ VG/ml. In one embodiment, the intraparenchymal (IPa), e.g., spinal cord, infusion is 1.8×10¹³ VG/ml, 1.85×10¹³ VG/ml, 1.9×10¹³ VG/ml, 1.95×10¹³ VG/ml, 2×10¹³ VG/ml, 2.01×10¹³ VG/ml, 2.02×10¹³ VG/ml, 2.03×10¹³ VG/ml, 2.04×10¹³ VG/ml, 2.05×10¹³ VG/ml, 2.06×10¹³ VG/ml, 2.07×10¹³ VG/ml, 2.08×10¹³ VG/ml, 2.09×10¹³ VG/ml, or 2.10×10¹³ VG/ml.

In one embodiment, the dose volume is 5 uL-60 uL per site of administration and the dose is 1.0×10¹⁰ VG-1.0×10¹² VG. In one embodiment, the dose volume is 5 uL-60 uL per site of administration and the dose is 5.0×10¹¹ VG-8.0×10¹¹ VG. In another embodiment, the dose volume is 25 uL-40 uL per site of administration and the dose is 1.0×10¹⁰ VG-1.0×10¹² VG. In another embodiment, the dose volume is 25 uL-40 uL per site of administration and the dose is 5.0×10¹¹ VG-8.0×10¹¹ VG. In one embodiment, the dose volume is 5 uL-60 uL for administration to level C3, C5, C6, or C7 of the spinal cord and the dose is 1.0×10¹⁰ VG-1.0×10¹² VG. In one embodiment, the dose volume is 5 uL-60 uL for administration to level C3, C5, C6, or C7 of the spinal cord and the dose is 5.0×10¹¹ VG-8.0×10¹¹ VG. In one embodiment, the dose volume is 5 uL-60 uL for administration to level C3 of the spinal cord and the dose is 1.0×10¹⁰ VG-1.0×10¹² VG. In one embodiment, the dose volume is 5 uL-60 uL for administration to level C3 of the spinal cord and the dose is 5.0×10¹¹ VG-8.0×10¹¹ VG. In another embodiment, the dose volume is 5 uL-60 uL for administration to level C5 of the spinal cord and the dose is 1.0×10¹⁰ VG-1.0×10¹² VG. In another embodiment, the dose volume is 5 uL-60 uL for administration to level C5 of the spinal cord and the dose is 5.0×10¹¹ VG-8.0×10¹¹ VG. In yet another embodiment; i) the dose volume is 5 uL-60 uL for administration to level C3 of the spinal cord and the dose is 1.0×10¹⁰ VG-1.0×10¹² VG, for example, 5.0×10¹¹ VG-8.0×10¹¹ VG, and ii) the dose volume for administration to level C5 of the spinal cord is 5 uL-60 uL and the dose is 1.0×10¹⁰ VG-1.0×10¹² VG, for example, 5.0×10¹¹ VG-8.0×10¹¹ VG. In one embodiment, the dose volume is 25 uL-40 uL for administration to level C3, C5, C6, or C7 of the spinal cord and the dose is 1.0×10¹⁰ VG-1.0×10¹² VG. In one embodiment, the dose volume is 25 uL-40 uL for administration to level C3, C5, C6, or C7 of the spinal cord and the dose is 5.0×10¹¹ VG-8.0×10¹¹ VG. In one embodiment, the dose volume is 25 uL-40 uL for administration to level C3 of the spinal cord and the dose is 1.0×10¹⁰ VG-1.0×10¹² VG. In one embodiment, the dose volume is 25 uL-40 uL for administration to level C3 of the spinal cord and the dose is 5.0×10¹¹ VG-8.0×10¹¹ VG. In another embodiment, the dose volume is 25 uL-40 uL for administration to level C5 of the spinal cord and the dose is 1.0×10¹⁰VG-1.0×10¹² VG. In another embodiment, the dose volume is 25 uL-40 uL for administration to level C5 of the spinal cord and the dose is 5.0×10¹¹ VG-8.0×10¹¹ VG. In yet another embodiment, i) the dose volume is 25 uL-40 uL for administration to level C3 of the spinal cord, and the dose is 1.0×10¹⁰ VG-1.0×10¹² VG, for example, 5.0×10¹¹ VG-8.0×10¹¹ VG, and ii) the dose volume for administration to level C5 of the spinal cord is 25 uL-40 uL, and the dose is 1.0×10¹⁰ VG-1.0×10¹² VG, for example, 5.0×10¹¹ VG-8.0×10¹¹ VG.

In one embodiment, the AAV particle described herein encoding siRNA molecules may be administered via intraparenchymal (IPa) infusion at two sites. The AAV particles may be delivered at the same or different volume for both sites. The AAV particles may be delivered at the same or different volumes for both sites. The AAV particles may be delivered at the same or different infusion rates for both sites.

In one embodiment, the AAV particle described herein encoding siRNA molecules may be administered via intraparenchymal (IPa) infusion at two sites. The AAV particles may be delivered at the same volume for both sites. The AAV particles may be delivered at the same dose for both sites. The AAV particles may be delivered at the same infusion rates for both sites.

In one embodiment, the AAV particle described herein encoding siRNA molecules may be administered via intraparenchymal (IPa) infusion at two sites. The AAV particles may be delivered at different volumes for both sites. The AAV particles may be delivered at different doses for both sites. The AAV particles may be delivered at different infusion rates for both sites.

In one embodiment, the AAV particle described herein encoding siRNA molecules may be administered via intraparenchymal (IPa) infusion at two sites. The AAV particles may be delivered at the same volume for both sites. The AAV particles may be delivered at different dose for both sites. The AAV particles may be delivered at different infusion rates for both sites.

In one embodiment, the AAV particle described herein encoding siRNA molecules may be administered via intraparenchymal (IPa) infusion at two sites. The AAV particles may be delivered at the same volume for both sites. The AAV particles may be delivered at different dose for both sites. The AAV particles may be delivered at the same infusion rates for both sites.

In one embodiment, the AAV particle described herein encoding siRNA molecules may be administered via intraparenchymal (IPa) infusion at two sites. The AAV particles may be delivered at the same volume for both sites. The AAV particles may be delivered at the same dose for both sites. The AAV particles may be delivered at different infusion rates for both sites.

In one embodiment, the AAV particle described herein encoding siRNA molecules may be administered via intraparenchymal (IPa) infusion at two sites. The AAV particles may be delivered at different volumes for both sites. The AAV particles may be delivered at the same dose for both sites. The AAV particles may be delivered at the same infusion rates for both sites.

In one embodiment, the AAV particle described herein encoding siRNA molecules may be administered via intraparenchymal (IPa) infusion at two sites. The AAV particles may be delivered at different volume for both sites. The AAV particles may be delivered at different dose for both sites. The AAV particles may be delivered at the same infusion rates for both sites.

In one embodiment, the AAV particle described herein encoding siRNA molecules may be administered via intraparenchymal (IPa) infusion at two sites. The AAV particles may be delivered at different volumes for both sites. The AAV particles may be delivered at the same dose for both sites. The AAV particles may be delivered at different infusion rates for both sites.

In one embodiment, the AAV particle described herein encoding siRNA molecules may be administered via intraparenchymal (IPa) infusion at C3 and C5. For the infusion at C3, the volume may be 25 uL and the dose may be 4.1×10¹¹ vg. For the infusion at C5, the volume may be 40 uL and the dose may be 6.6×10¹¹ vg. The injection rate for both infusions may be 5 uL/min for about 13 minutes.

In some embodiments, IPa infusions (e.g., spinal cord) may result in delivery of the pharmaceutical compositions (i.e., AAV particles) along the extent of the rostral-caudal axis of the spinal cord. In some embodiments, IPa infusions (e.g., spinal cord) yield a rostrocaudal gradient of AAV particle transmission. In some embodiments, IPa infusions (e.g., spinal cord) result in delivery of the pharmaceutical compositions to regions distal to the injection site. While not wishing to be bound by theory, AAV particles of the disclosure may travel the length of the rostral caudal axis of the spinal cord subsequent to IPa infusion at a particular site. In other words, the AAV particles may not confined to the immediate vicinity of the injection site. As a non-limiting example, the AAV particles may be transported by a trans-synaptic (across the synapse) mechanism. This trans-synaptic mechanism may follow a tract or channel present along the rostral-caudal axis of the spinal cord.

The pharmaceutical compositions of the present disclosure may be administered to a subject using any amount effective for preventing and treating a SOD1 associated disorder (e.g., ALS). The exact amount required will vary from subject to subject, depending on the species, age, and general condition of the subject, the severity of the disease, the particular composition, its mode of administration, its mode of activity, and the like.

The compositions of the present disclosure are typically formulated in unit dosage form for ease of administration and uniformity of dosage. It will be understood, however, that the total daily usage of the compositions of the present disclosure may be decided by the attending physician within the scope of sound medical judgment. The specific therapeutically effectiveness for any particular patient will depend upon a variety of factors including the disorder being treated and the severity of the disorder; the activity of the specific compound employed; the specific composition employed; the age, body weight, general health, sex and diet of the patient; the time of administration, and route of administration; the duration of the treatment; drugs used in combination or coincidental with the specific compound employed; and like factors well known in the medical arts.

In some specific embodiments, the doses of AAV vectors for delivering siRNA duplexes of the present disclosure may be adapted dependent on the disease condition, the subject and the treatment strategy, etc. Typically, about 10⁵, 10⁶, 10¹², 10¹³, 10¹⁴, 10¹⁵ to 10¹⁶ viral genome (unit) may be administered per dose.

The desired dosage may be delivered three times a day, two times a day, once a day, every other day, every third day, every week, every two weeks, every three weeks, or every four weeks.

In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. As used herein, a “split dose” is the division of single unit dose or total daily dose into two or more doses, e.g., two or more administrations of the single unit dose. As used herein, a “single unit dose” is a dose of any modulatory polynucleotide therapeutic administered in one dose/at one time/single route/single point of contact, i.e., single administration event. As used herein, a “total daily dose” is an amount given or prescribed in 24 hr period. It may be administered as a single unit dose. In one embodiment, the viral vectors comprising the SOD1 targeting polynucleotides of the present disclosure are administered to a subject in split doses. They may be formulated in buffer only or in a formulation described herein.

### V. Definitions

Unless stated otherwise, the following terms and phrases have the meanings described below. The definitions are not meant to be limiting in nature and serve to provide a clearer understanding of certain aspects of the present disclosure.

As used herein, a “cannula” refers to a small tube for insertion into a body cavity (natural or surgically created) or into a duct or vessel of a subject. For clarity the terms “tube” or “delivery tube” or “cannula” may be used interchangeably. The cannula tube can be made from one or more medical-grade materials including stainless steel, titanium, polyvinylchloride (PVC), polymethylmethacrylate (PMMA), polytetrafluoroethylene (PTFE) or a fused silica such as quartz silica. In certain embodiments, the tube is made from a flexible material. In certain embodiments, the tube includes a polymeric coating, such as a polyimide coating. In certain embodiments, the tube has a diameter and thickness which allow the tube to be flexible.

As used herein, the term “tissue” refers to a collection of interconnected cells or biological materials that perform a similar function within an organism. Accordingly, the term “tissue” as used herein broadly encompasses all biological components including, but not limited to, skin, muscle, nerves, blood, bone, cartilage, teeth, tendons, ligaments, and organs composed of or containing the same, as well as corresponding biological materials and fluids such as cerebrospinal fluids. The term “tissue” can include parenchymal tissue and/or non-parenchymal tissue.

As used herein, the term “nucleic acid”, “polynucleotide” and “oligonucleotide” refer to any nucleic acid polymers composed of either polydeoxyribonucleotides (containing 2-deoxy-D-ribose), or polyribonucleotides (containing D-ribose), or any other type of polynucleotide which is an N glycoside of a purine or pyrimidine base, or modified purine or pyrimidine bases. There is no intended distinction in length between the term “nucleic acid”, “polynucleotide” and “oligonucleotide”, and these terms will be used interchangeably. These terms refer only to the primary structure of the molecule. Thus, these terms include double- and single-stranded DNA, as well as double- and single stranded RNA.

As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

As used herein, the term “small/short interfering RNA” or “siRNA” refers to an RNA molecule (or RNA analog) comprising between about 5-60 nucleotides (or nucleotide analogs) which is capable of directing or mediating RNAi. Preferably, a siRNA molecule comprises between about 15-30 nucleotides or nucleotide analogs, more preferably between about 16-25 nucleotides (or nucleotide analogs), even more preferably between about 18-23 nucleotides (or nucleotide analogs), and even more preferably between about 19-22 nucleotides (or nucleotide analogs) (e.g., 19, 20, 21 or 22 nucleotides or nucleotide analogs). The term “short” siRNA refers to a siRNA comprising 5-23 nucleotides, preferably 21 nucleotides (or nucleotide analogs), for example, 19, 20, 21 or 22 nucleotides. The term “long” siRNA refers to a siRNA comprising 24-60 nucleotides, preferably about 24-25 nucleotides, for example, 23, 24, 25 or 26 nucleotides. Short siRNAs may, in some instances, include fewer than 19 nucleotides, e.g., 16, 17 or 18 nucleotides, or as few as 5 nucleotides, provided that the shorter siRNA retains the ability to mediate RNAi. Likewise, long siRNAs may, in some instances, include more than 26 nucleotides, e.g., 27, 28, 29, 30, 35, 40, 45, 50, 55, or even 60 nucleotides, provided that the longer siRNA retains the ability to mediate RNAi or translational repression absent further processing, e.g., enzymatic processing, to a short siRNA, siRNAs can be single stranded RNA molecules (ss-siRNAs) or double stranded RNA molecules (ds-siRNAs) comprising a sense strand and an antisense strand which hybridized to form a duplex structure called siRNA duplex. According to the present disclosure, recombinant AAV vectors may encode one or more RNAi molecules such as an siRNA, shRNA, microRNA or precursor thereof.

As used herein, the term “the antisense strand” or “the first strand” or “the guide strand” of a siRNA molecule refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process.

As used herein, the term “the sense strand” or “the second strand” or “the passenger strand” of a siRNA molecule refers to a strand that is complementary to the antisense strand or first strand. The antisense and sense strands of a siRNA molecule are hybridized to form a duplex structure. As used herein, a “siRNA duplex” includes a siRNA strand having sufficient complementarity to a section of about 10-50 nucleotides of the mRNA of the gene targeted for silencing and a siRNA strand having sufficient complementarity to form a duplex with the siRNA strand. According to the present disclosure, recombinant AAV vectors may encode a sense and/or antisense strand.

As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pair in the Watson-Crick manner (e.g., A to T. A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present disclosure, the ability to substitute a T is implied, unless otherwise stated. Perfect complementarity or 100% complementarity refers to the situation in which each nucleotide unit of one polynucleotide strand can form hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form hydrogen bond with each other, the polynucleotide strands exhibit 10% complementarity. In the same example, if 18 base pairs on each strand can form hydrogen bonds with each other, the polynucleotide strands exhibit 90% complementarity.

As used herein. “targeting” means the process of design and selection of nucleic acid sequence that will hybridize to a target nucleic acid and induce a desired effect.

The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

As used herein, the term “mutation” refers to any changing of the structure of a gene, resulting in a variant (also called “mutant”) form that may be transmitted to subsequent generations. Mutations in a gene may be caused by the alternation of single base in DNA, or the deletion, insertion, or rearrangement of larger sections of genes or chromosomes.

As used herein, the term “vector” means any molecule or moiety which transports, transduces or otherwise acts as a carrier of a heterologous molecule such as the SOD1 targeting polynucleotides of the disclosure. A “viral vector” is a vector which comprises one or more polynucleotide regions encoding or comprising a molecule of interest, e.g., a transgene, a polynucleotide encoding a polypeptide or multi-polypeptide or a modulatory nucleic acid such as small interfering RNA (siRNA). Viral vectors are commonly used to deliver genetic materials into cells. Viral vectors are often modified for specific applications. Types of viral vectors include retroviral vectors, lentiviral vectors, adenoviral vectors and adeno-associated viral vectors.

The terms “adeno-associated virus”. “AAV”, “AAV vector”, “AAV particle”, or “AAV vector particle” as used herein refers to any vector or particle which comprises or derives from components of an adeno associated vector and is suitable to infect mammalian cells, preferably human cells. An AAV particle typically includes an AAV vector genome encased within an AAV capsid shell. The term AAV vector typically designates an AAV type viral particle or virion comprising a nucleic acid molecule encoding a siRNA duplex. The AAV vector may be derived from various serotypes, including combinations of serotypes (i.e., “pseudotyped” AAV) or from various genomes (e.g., single stranded or self-complementary). In addition, the AAV vector may be replication defective and/or targeted.

As used herein, the phrase “inhibit expression of a gene” means to cause a reduction in the amount of an expression product of the gene. The expression product can be a RNA molecule transcribed from the gene (e.g., an mRNA) or a polypeptide translated from an mRNA transcribed from the gene. Typically, a reduction in the level of an mRNA results in a reduction in the level of a polypeptide translated therefrom. The level of expression may be determined using standard techniques for measuring mRNA or protein.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe or cell or tissue thereof).

As used herein, the term “modified” refers to a changed state or structure of a molecule of the disclosure. Molecules may be modified in many ways including chemically, structurally, and functionally.

As used herein, the term “synthetic” means produced, prepared, and/or manufactured by the hand of man. Synthesis of polynucleotides or polypeptides or other molecules of the present disclosure may be chemical or enzymatic.

As used herein, the phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “therapeutic agent” refers to a substance which has a therapeutic effect on the health and well-being of a target subject when administered in a therapeutically effective amount. The therapeutic effect can include: preventing, delaying or curing an ailment, disease or disorder; mitigating, slowing, pausing or reversing the progress of an ailment, disease or disorder; or alleviating or reducing one or more symptom associated with an ailment, disease or disorder.

As used herein, the term “effective amount” of an agent is that amount sufficient to effect beneficial or desired results, for example, clinical results, and, as such, an “effective amount” depends upon the context in which it is being applied. For example, in the context of administering an agent that treats ALS, an effective amount of an agent is, for example, an amount sufficient to achieve treatment, as defined herein, of ALS, as compared to the response obtained without administration of the agent.

As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

As used herein, the term “subject” or “patient” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates such as chimpanzees and other apes and monkey species, and humans) and/or plants.

As used herein, the term “preventing” or “prevention” refers to delaying or forestalling the onset, development or progression of a condition or disease for a period of time, including weeks, months, or years.

The term “treatment” or “treating”, as used herein, refers to the application of one or more specific procedures used for the cure or amelioration of a disease. In certain embodiments, the specific procedure is the administration of one or more pharmaceutical agents.

As used herein, the term “amelioration” or “ameliorating” refers to a lessening of severity of at least one indicator of a condition or disease. For example, in the context of neurodegeneration disorder, amelioration includes the reduction of neuron loss.

As used herein, the term “administering” refers to providing a pharmaceutical agent or composition to a subject.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

### EXAMPLES

### Example 1. Intraparenchymal Delivery of SOD1 siRNA to Spinal Cord

Three Gottingen adult (6 months of age), female mini-pigs weighing 14-20 kg each were utilized for each of the groups in the study. Animals were not pre-screened for neutralizing antibodies to AAV. A 4-5 cm laminectomy was performed between C3 and C5, allowing for 3 cm between injections. Self-complementary (sc) AAV vector (scAAV) with a modulatory polynucleotide (SEQ ID NO: 1) comprising siRNA targeting SOD1 and an ITR to ITR sequence (SEQ ID NO: 3) which includes an albumin derived filler sequence were packaged into AAVrh10 vector to generate scAAVrh10.H1.mir104-788.2 (albumin).

Animals were injected with one of two doses using a cannula system of the present invention which includes an even transition of therapeutic agent through the lumen of a fused silica delivery tube to a 27 gage stainless steel hypodermic needle tip. For the higher of the two doses, two injections of the scAAV (titer 1.73×10¹³ vg/mL) were administered. At the rostral end of the laminectomy, i.e. at the C3 level of the spinal cord, a single 40 μL (6.9×10¹¹ vg/injection) volume was injected into the ventral horn of the spinal cord. At the caudal end of the laminectomy, i.e. at the C5 level of the spinal cord, a single 40 μL (6.9×10¹¹ vg/injection) volume was injected into the ventral horn of the contralateral side. For the lower of the two doses, two injections of the scAAV (titer 5.8×10¹¹ vg/mL, 1/30^{th} dose of 1.73×10¹³ vg/mL) were administered. At the rostral end of the laminectomy, i.e. at the C3 level of the spinal cord, a single 40 μL (2.3×10¹⁰ vg/injection) volume was injected into the ventral horn of the spinal cord. At the caudal end of the laminectomy, i.e. at the C5 level of the spinal cord, a single 40 μL (2.3×10¹⁰ vg/injection) volume was injected into the ventral horn of the contralateral side. All injections were administered at the rate of 5 μL/min, yielding an approximately 13-minute total infusion time. Four weeks following the procedure, animals were sacrificed, and spinal cord tissue was collected for analyses.

### Example 2. Intraparenchymal Delivery Using Adhesive

A C3-C5 laminectomy was performed by making a small incision in the cervical spinal area of a subject and displacing muscles and soft tissue, thereby exposing the C3, C4 and C5 vertebral bones. A C4 bilateral infusion (120 μL) was performed by placing cannula systems of the present invention (fused silica delivery tip, blunt ended) on both the right and left side of the C4 target area. An Evicel adhesive was administered to secure both cannula systems to the target. Therapeutic agent was delivered through each cannula, and the systems were then removed. Almost all Evicel was able to be removed after the cannulas were removed.

We claim:

1. A cannula system comprising a proximal region and a distal region, and further comprising a delivery tube which extends from the proximal region of the cannula system to the distal region of the cannula system.

2. The cannula system of claim 1, wherein the delivery tube is be made from a medical-grade material.

3. The cannula system of claim 1 or 2, wherein the delivery tube is made from a material selected from: stainless steel, titanium, polyvinylchloride (PVC), polymethyl methacrylate (PMMA) and polytetrafluoroethylene (PTFE).

4. The cannula system of claim 1 or 2, wherein the delivery tube is made from a fused silica such as quartz silica.

5. The cannula system of claim 1 or 2, wherein the delivery tube is made from stainless steel.

6. The cannula system of any one of claims 1-5, wherein the delivery tube has an inner diameter of 200 μm, 250 μm, 300 μm, or between 200-300 μm.

7. The cannula system of any one of claims 1-6, wherein the delivery tube comprises a delivery tip at the distal end of the delivery tube.

8. The cannula system of claim 7, wherein the delivery tip is made from stainless steel.

9. The cannula system of any one of claims 7-8, wherein the delivery tip comprises one or more delivery tip openings.

10. The cannula system of any one of claims 7-9, wherein the delivery tip is a blunt tip, a flat tip, a conic tip, a needle tip, a bevel tip or an angled tip.

11. The cannula system of any one of claims 1-10, wherein the distal region of the cannula system comprises a rigid tube element.

12. The cannula system of claim 11, wherein the rigid tube element has a length of 9.0-11.0 mm.

13. The cannula system of claim 12, wherein the rigid tube element has a length of 10.0 mm.

14. The cannula system of any one of claims 11-13, wherein the rigid tube has an inner diameter of 750 μm, 800 μm, 850 μm, or between 750-850 μm.

15. The cannula system of any one of claims 11-14, wherein the rigid tube has an outer diameter of 2.0 mm, 2.5 mm, 3.0 μm, or between 2.0-3.0 mm.

16. The cannula system of any one of claims 11-15, wherein the rigid tube element comprises a proximal end and a distal end, and further comprises a rigid tube channel extending through the rigid tube for the proximal rigid tube end to the distal rigid tube end; and wherein the delivery tube extends through the rigid tube channel of the rigid tube element, and wherein the delivery tube is longer than the rigid tube element such that a portion of the distal end of the delivery tube extends beyond the distal end of the rigid tube to form an exposed delivery tube element.

17. The cannula system of claim 16, wherein the exposed delivery tube element at the distal end of the delivery tube has a length of between 2.5-5.0 mm.

18. The cannula system of claim 16, wherein the exposed delivery tube element at the distal end of the delivery tube has a length of between 3.0-3.5 mm.

19. The cannula system of claim 16, wherein the exposed delivery tube element at the distal end of the delivery tube has a length of between 3.5-4.0 mm.

20. The cannula system of claim 16, wherein the exposed delivery tube element at the distal end of the delivery tube has a length of between 4.0-4.5 mm.

21. The cannula system of claim 16, wherein the exposed delivery tube element at the distal end of the delivery tube has a length of between 4.5-5.0 mm.

22. The cannula system of any one of claims 16-21, wherein the distal region of the cannula system comprises a capillary tube which comprises a capillary tube channel extending through the capillary tube from a distal end of the capillary tube to a proximal end of the capillary tube; wherein the capillary tube extends through the rigid tube channel; and wherein the delivery tube extends through the capillary tube channel.

23. The cannula system of claim 22, wherein the capillary tube has an inner diameter which is sufficiently small to prevent liquid from flowing between the capillary tube and the delivery tube; and wherein the capillary tube has an outer diameter which is sufficiently large to prevent liquid from flowing between the capillary tube and the rigid tube element.

24. The cannula system of any one of claims 11-23, wherein the rigid tube element is made from a material selected from: stainless steel, titanium, polyvinylchloride (PVC), polymethyl methacrylate (PMMA), and polytetrafluoroethylene (PTFE).

25. The cannula system of any one of claims 11-23, wherein the rigid tube element is made from a ceramic material.

26. The cannula system of any one of claims 11-23, wherein the rigid tube element is made from an alumina-based ceramic material or a zirconia-based ceramic material.

27. The cannula system of any one of claims 11-26, wherein the cannula system comprises a support tube which extends from the proximal region of the cannula system to the distal region of the cannula system; wherein the support tube comprises a support tube channel extending through the support tube from a distal end of the support tube to a proximal end of the support tube; and wherein the delivery tube extends through the support tube channel from the proximal region of the cannula system to the distal region of the cannula system.

28. The cannula system of claim 27, wherein the support tube is made from a material selected from: stainless steel, titanium, polyvinylchloride (PVC), polymethyl methacrylate (PMMA), and polytetrafluoroethylene (PTFE).

29. The cannula system of any one of claims 27-28, wherein the distal end of the support tube is adjacent to or in contact with the proximal end of the rigid tube element.

30. The cannula system of any one of claims 27-28, wherein a portion of the distal end of the rigid tube element extends into the support tube channel at the proximal end of the support tube.

31. The cannula system of any one of claims 29-30, wherein the cannula system comprises a collar tube which extends over a portion of the distal end of the support tube and extends over a portion of the proximal end of the rigid tube element.

32. The cannula system of any one of claims 1-31, wherein the cannula system comprises a cannula positioning guide (CPG), said CPG comprising:
a guide base comprising an upper surface, a lower surface, and at least one edge surface, said guide base being suitable for connecting, appending or adhering the CPG directly or indirectly to a target tissue; and
at least one comb projection extending from the edge surface of the guide base.

33. The cannula system of claim 32, wherein the CPG further comprises a first pedicle and a second pedicle, with both pedicles being positioned on the upper or lower surface of the base.

34. The cannula system of claim 33, wherein the CPG further comprises an alignment fence juxtaposed between the first pedicle and the second pedicle.

35. The cannula system of any one of claims 32-34, wherein the CPG further comprises one or more clamps, clips or attachment devices.

36. The cannula system of any one of claims 32-35, wherein the CPG is made from medical-grade materials.

37. The cannula system of claim 36, wherein the medical-grade material of the CPG is suitable for use during magnetic resonance imaging (MRI).

38. The cannula system of any one of claims 32-37, wherein the CPG further comprises an illumination device.

39. A kit comprising: (i) the cannula system of any one of claims 1-38, and (ii) an adhesive.

40. The kit of claim 39, wherein the adhesive comprises DuraSeal, DuraSeal Exact, Adherus, or Evicel.

41. A method of delivering a therapeutic agent to a therapeutic target tissue in a subject, comprising: (i) providing a cannula system of any one of claims 1-38; (ii) inserting a portion of the delivery tube of the cannula system into a delivery target tissue, wherein the delivery target tissue is the therapeutic target tissue or corresponding tissue which facilitates the delivery of the therapeutic agent to the therapeutic target tissue; and (iii) delivering a therapeutically effective amount of the therapeutic agent through the delivery tube of the cannula system into the delivery target tissue.

42. The method of claim 41, wherein the delivery target tissue is parenchymal tissue in the brain of the subject.

43. The method of claim 41, wherein the delivery target tissue is parenchymal tissue in the eye or heart of the subject.

44. The method of claim 41, wherein the delivery target tissue is parenchymal tissue in the spine of the subject.

45. The method of claim 41, wherein the delivery target tissue is non-parenchymal tissue in the spine of the subject.

46. The method of claim 41, wherein the therapeutic target tissue is parenchymal tissue in the spine of the subject; and wherein the delivery target tissue is non-parenchymal tissue in the spine which is adjacent to or corresponds with the parenchymal tissue, and which facilitates the delivery of the therapeutic agent to the parenchymal tissue in the spine.

47. The method of claim 41, wherein the delivery target tissue is selected from: the posterior white column, the lateral white column, the anterior white column, the posterior gray horn, the lateral gray horn, the posterior gray commissure, the anterior gray commissure, the anterior white commissure, the central canal, the ventral horn, and combinations thereof.

48. The method of any one of claims 41-47, wherein the therapeutic agent comprises a small molecule compound, a naturally-occurring pharmaceutical compound, a synthetic pharmaceutical compound, a polypeptide, a protein, a polynucleotide, a viral vector, a biological progenitor cells, a stem cell, a biological transplant cell, a macrophage, a T cell, or a combination thereof.

49. The method of any one of claims 41-47, wherein the therapeutic agent comprises an RNAi molecule.

50. The method of any one of claims 41-47, wherein the therapeutic agent comprises an AAV vector particle which comprises an AAV capsid and an AAV vector genome, wherein the AAV vector genome comprises a nucleotide sequence encoding an RNAi molecule.

51. The method of any one of claims 49-50, wherein the RNAi molecule is an siRNA strand or an siRNA duplex.

52. The method of any one of claims 49-51, wherein the RNAi molecule is an SOD1 targeting polynucleotide.

53. The method of any one of claims 41-52, wherein the target subject has amyotrophic lateral sclerosis (ALS).
